(19) United States
(12) Patent Application Publication
(10) Pub. No.: US 2012/0096381 A1
(43) Pub. Date: Apr. 19, 2012

(54) Ventilator-Initiated Prompt In Response To Proposed Setting Adjustment

(75) Inventors: Gary Milne, Louisville, CO (US); Kirk Hensley, Dublin, OH (US); Peter R. Doyle, Vista, CA (US); Gardner Kimm, Carlsbad, CA (US)

(73) Assignee: Nellcor Puritan Bennett LLC, Boulder, CO (US)

(21) Appl. No.: 12/903,358

(22) Filed: Oct. 13, 2010

(51) Int. Cl. G06F 3/048; A61M 16/00

(52) U.S. Cl. 715/771; 128/204.21; 715/835

(57) ABSTRACT

This disclosure describes systems and methods for issuing a prompt in response to one or more proposed settings adjustments. Specifically, the prompt may include a projected impact of the one or more proposed settings adjustments on patient condition and/or patient treatment. The prompt may further provide an impact level and/or an alert associated with the projected impact. According to embodiments, one or more recommendations for alternative settings adjustments may be provided on the prompt or on an extension of the prompt. According to embodiments, a clinician may scroll through a plurality of potential settings adjustments until a desired impact level is displayed on the prompt (e.g., a positive impact level). According to still other embodiments, a plurality of proposed settings adjustments may be received. In this case, the projected impact displayed on the prompt may represent a combined projected impact of the plurality of proposed settings adjustments received.

### INTRODUCTION

A ventilator is a device that mechanically helps patients breathe by replacing some or all of the muscular effort required to inflate and deflate the lungs. In recent years, there has been an accelerated trend towards an integrated clinical environment. That is, medical devices are becoming increasingly integrated with communication, computing, and control technologies. As a result, modern ventilatory equipment has become increasingly complex, providing for detection, monitoring, and evaluation of a myriad of ventilatory parameters during ventilation of a patient. However, even in light of available ventilatory data, many clinicians may not readily assess and evaluate the projected impact of a proposed setting adjustment on patient condition.

Indeed, clinicians and patients may greatly benefit from ventilator notifications identifying how proposed settings adjustments may impact patient condition, either positively or negatively, based on evaluation of available physiological and ventilatory data for the patient.

Ventilator-Initiated Prompt in Response to Proposed Setting Adjustment

This disclosure describes systems and methods for issuing a prompt in response to one or more proposed settings adjustments. The prompt may be configured to provide valuable information to a clinician regarding how a proposed setting adjustment may impact patient condition and/or patient treatment. Specifically, the prompt may include a projected impact of the one or more proposed settings adjustments on patient condition and/or patient treatment. The projected impact may be calculated based on, inter alia, current ventilatory settings, collected ventilatory data, patient data, and/or any other suitable protocol, equation, etc. The prompt may further provide an impact level and/or an alert associated with the projected impact. According to embodiments, one or more recommendations for alternative settings adjustments may be provided on the prompt or on an extension of the prompt. The alternative settings adjustments may be calculated based on, inter alia, current ventilatory settings, collected ventilatory data, patient data, and/or any other suitable protocol, equation, etc. According to other embodiments, the prompt may provide an impact level upon entry of a proposed setting adjustment such that a clinician may scroll through a plurality of potential settings adjustments until a desired impact level is displayed on the prompt (e.g., a positive or green impact level). According to still other embodiments, a plurality of proposed settings adjustments may be received. In this case, the projected impact displayed on the prompt may represent a combined projected impact of the plurality of proposed settings adjustments received.

According to embodiments, a ventilator-implemented method is provided for issuing a prompt in response to a proposed setting adjustment during ventilation of a patient. The method may comprise receiving the proposed setting adjustment and retrieving a patient diagnosis, at least some collected ventilatory data, and one or more current ventilatory settings. The method may further comprise determining and displaying a projected impact of the proposed setting adjustment based on the one or more current ventilatory settings, the patient diagnosis, and the at least some collected ventilatory data.

According to additional embodiments, a ventilatory system is provided for issuing a prompt in response to a proposed ventilatory setting adjustment during ventilation of a patient. The ventilator system may be configured to receive a proposed setting adjustment and to retrieve at least some collected ventilatory data and one or more current ventilatory settings. The ventilator system may be further configured to determine a projected impact of the proposed setting adjustment based on the one or more current ventilatory settings and the at least some collected ventilatory data. The ventilator system may also determine one or more alternative settings adjustments, comprising identifying a target ventilation change and determining one or more alternative settings adjustments for achieving the target ventilation change based on the one or more current ventilatory settings and the at least some collected ventilatory data. The ventilator system may be further configured to display the prompt identifying the projected impact of the proposed setting adjustment and the one or more alternative settings adjustments.

According to further embodiments, a graphical user interface is provided for displaying one or more prompts in response to receiving a proposed setting adjustment. The graphical user interface may comprise at least one window and one or more elements within the at least one window comprising at least one prompt element. The at least one prompt element may communicate information regarding the proposed setting adjustment, including identifying a projected impact of the proposed setting adjustment.

According to still further embodiments, a ventilator processing interface is provided for displaying one or more prompts in response to receiving a proposed setting adjustment. The ventilator processing interface may comprise means for accepting the proposed setting adjustment and means for retrieving one or more current ventilatory settings and at least some collected ventilatory data. Further, the ventilator processing interface may comprise means for determining a projected impact of the proposed setting adjustment and means for displaying a prompt comprising the projected impact.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the claims.

### BRIEF DESCRIPTION OF THE DRAWINGS

The following drawing figures, which form a part of this application, are illustrative of described technology and are not meant to limit the scope of the claims in any manner, which scope shall be based on the claims appended hereto.

FIG. 1 is a diagram illustrating an embodiment of an exemplary ventilator connected to a human patient.

FIG. 2 is a block-diagram illustrating an embodiment of a ventilatory system for receiving one or more proposed settings adjustments, calculating a projected impact of the one or more proposed settings adjustments, and issuing an appropriate prompt.

FIG. 3 is a flow chart illustrating an embodiment of a method for issuing a prompt identifying a projected impact of a proposed setting adjustment based on a patient diagnosis and collected ventilatory data.

FIG. 4 is a flow chart illustrating an embodiment of a method for issuing a prompt identifying a projected impact of a proposed setting adjustment and suggesting one or more alternative settings adjustments.

FIG. 5 is a flow chart illustrating an embodiment of a method for issuing a prompt identifying a projected impact and an impact level of a proposed setting adjustment and suggesting one or more alternative settings adjustments.

FIG. 6 is an illustration of an embodiment of a graphical user interface displaying a prompt identifying a projected impact and a positive impact level of a proposed setting adjustment.

FIG. 7 is an illustration of an embodiment of a graphical user interface displaying a prompt identifying a projected impact having an alert and an intermediate impact level of a proposed setting adjustment.

FIG. 8 is an illustration of an embodiment of a graphical user interface displaying a prompt identifying a projected impact having an alert and a negative impact level of a proposed setting adjustment.

FIG. 9 is an illustration of an embodiment of a graphical user interface displaying a prompt identifying a combined projected impact having an alert and an intermediate impact level of a plurality of proposed settings adjustments.

FIG. 10 is an illustration of an embodiment of a graphical user interface displaying a prompt and an expanded prompt identifying a projected impact of a proposed setting adjustment and identifying one or more recommendations for alternative settings adjustments.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment for alerting and advising clinicians regarding a projected impact of a proposed settings change.

According to embodiments, the ventilator may be configured to collect ventilatory data by monitoring and evaluating diverse ventilatory parameters and/or patient physiological data. Based on the collected ventilatory data, the ventilator may issue suitable notifications and recommendations to the clinician upon receiving a proposed setting adjustment. That is, the ventilator may determine a projected impact on patient condition and/or ventilatory treatment of the proposed setting adjustment based on, infer alia, current ventilatory settings, the collected ventilatory data, and/or any suitable protocol, equation, etc. According to additional embodiments, the ventilator may receive patient data (e.g., a patient body weight, a patient diagnosis, a patient gender, age, a patient disability, a patient post-operative condition, etc.) in addition to the collected ventilatory data. In this case, the ventilator may determine the projected impact on patient condition and/or ventilatory treatment of the proposed setting adjustment based on, inter alia, current ventilatory settings, the collected ventilatory data, the patient data, and/or any suitable protocol, equation, etc.

According to other embodiments, the projected impact on patient condition and/or ventilatory treatment may be associated with a positive, a negative, or an intermediate impact level. The suitable notifications may be configured, e.g., graphically or otherwise, such that a projected positive impact level is identifiable in one format (e.g., green, normal typeface, “smiling face” icon, etc.), a projected negative impact level is identifiable in another format (e.g., red, bold typeface, “exclamation mark” or “frowning face” icon, etc.), and a projected intermediate impact level is identifiable in yet another format (e.g., yellow, italic typeface, “straight-faced” icon, etc.). According to some embodiments, a projected negative or red impact level may impose additional confirmatory steps by the clinician in order to accept the proposed setting adjustment. According to other embodiments, the prompt may provide the impact level as a proposed setting adjustment is entered such that a clinician may scroll through a plurality of potential settings adjustments until a desired impact level is displayed on the prompt, e.g., a positive or green impact level.

According to embodiments, in addition to a notification regarding a projected impact of a proposed setting adjustment, the ventilator may issue an alert associated with the projected impact. For example, the alert may caution the clinician regarding a potential patient condition that may result should the proposed setting adjustment be accepted (e.g., Auto-PEEP implicated).

According to other embodiments, the prompt may provide recommendations for one or more alternative settings adjustments. The ventilator may determine the one or more alternative settings adjustments by identifying a target ventilation change and then identifying one or more alternative settings adjustments for achieving the target ventilation change based on, inter alia, the current ventilatory settings, the collected ventilatory data, the patient data, and/or any suitable protocol, equation, etc. According to embodiments, the one or more alternative settings adjustments may be displayed on an initial prompt or, for example, may be optionally accessed by a clinician and displayed on an extension of the prompt. The prompt may further provide the one or more alternative settings adjustments such that they may be selectively activated and accepted by the clinician in lieu of the proposed setting adjustment.

According to still other embodiments, a plurality of proposed settings adjustments may be received. According to some embodiments, a prompt may issue upon entry of each of the plurality of proposed settings adjustments, such that the projected impact of each consecutive entry takes into consideration any previous proposed settings adjustments. That is, as each of the plurality of proposed settings adjustments is received, a combined projected impact may be provided (e.g., automatically or upon activation of a “review” button). According to other embodiments, a prompt may not issue until all of the plurality of proposed settings adjustments have been received (e.g., upon receipt of an indication that all of the plurality of proposed settings adjustments have been input). In such case, the prompt may issue a single projected impact that represents a combined projected impact of all of the plurality of proposed settings adjustments. According to still other embodiments, a prompt may not issue unless and until selected by a clinician. For example, a “check” or “review” control may be initiated after entering one or more proposed settings adjustments and the prompt may be displayed only upon activation of such control.

These and other embodiments will be discussed in further detail with reference to the following figures.

### Ventilator System

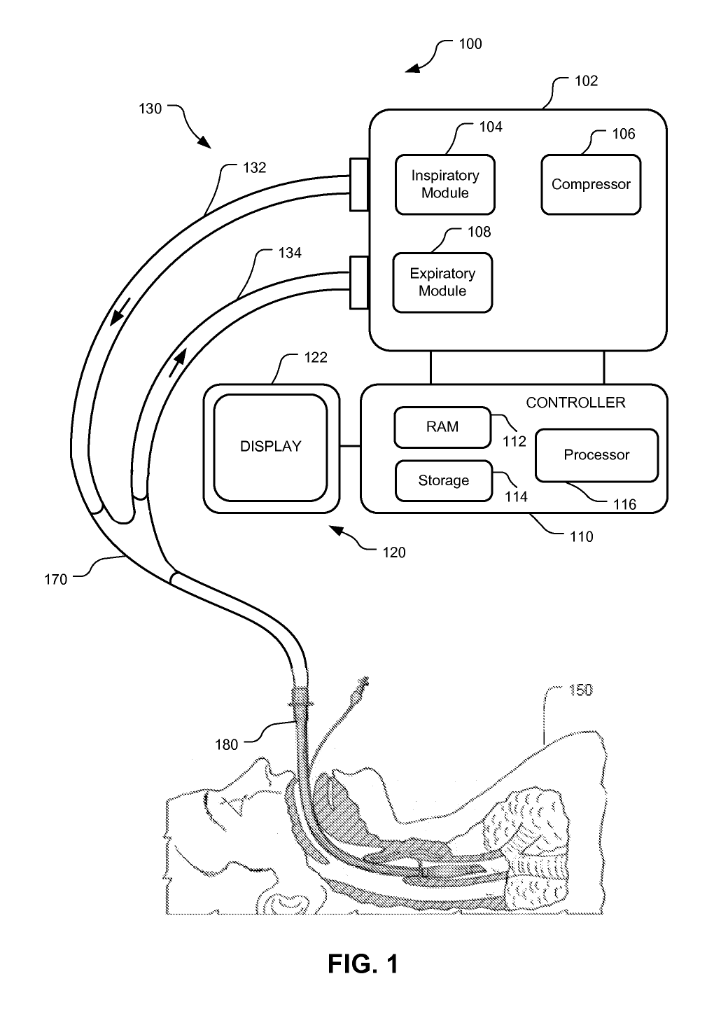

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.). Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display to serve both as an input and output device.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to one or more processors 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the one or more processors 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

### Ventilator Components

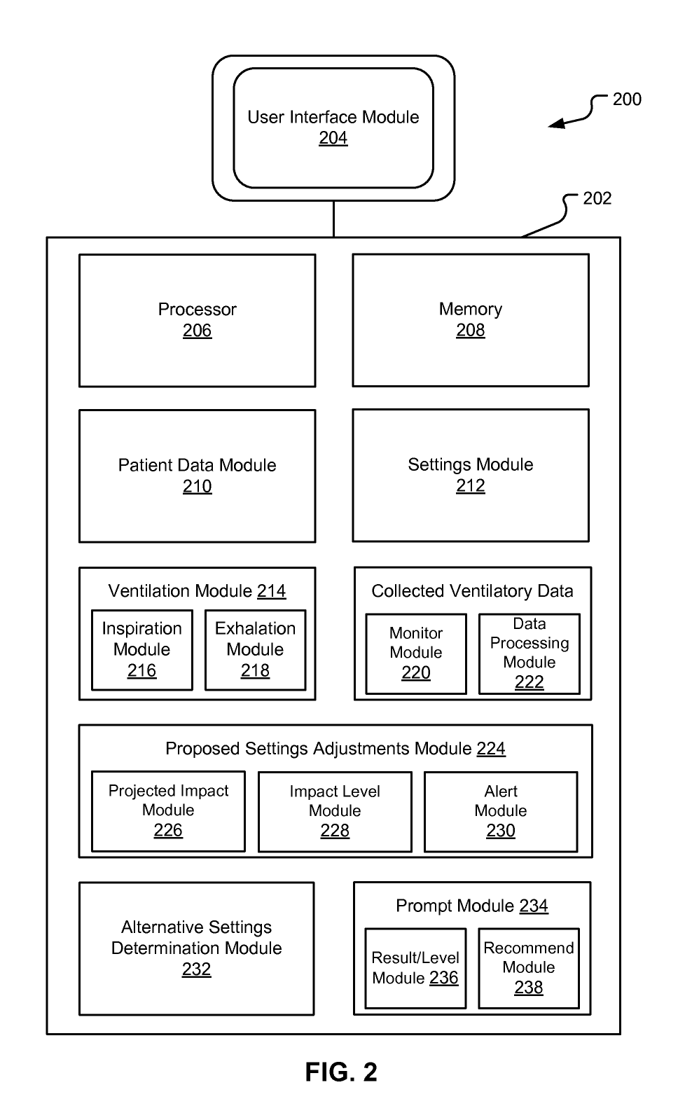

FIG. 2 is a block-diagram illustrating an embodiment of a ventilatory system for receiving one or more proposed settings adjustments, calculating a projected impact of the one or more proposed settings adjustments, and issuing an appropriate prompt.

Ventilatory system 200 includes ventilator 202 with its various modules and components. That is, ventilator 202 may further include, inter alga, memory 208, one or more processors 206, user interface module 204, and ventilation module 214 (which may further include an inspiration module 216 and an exhalation module 218). Memory 208 is defined as described above for memory 112. Similarly, the one or more processors 206 are defined as described above for one or more processors 116. Processors 206 may further be configured with a clock whereby elapsed time may be monitored by the system 200.

As described above, the ventilatory system 200 may also include user interface module 204 communicatively coupled to ventilator 202. User interface module 204 may provide various input screens, for receiving clinician input, and various display screens, for presenting useful information to the clinician. For example, the user interface module 204 may include a graphical user interface (GUI). The GUI may be an interactive display, e.g., a touch-sensitive screen or otherwise, and may provide various windows and elements for receiving input and interface command operations. Alternatively or additionally, other suitable means of communication with the ventilator 202 may be provided, for instance by a wheel, keyboard, mouse, or other suitable interactive device. The ventilator may receive ventilatory settings and adjustments to ventilatory settings via a settings module 212. Based on the ventilatory settings, the ventilator may provide ventilation to a patient via the ventilation module 214.

User interface module 204 may also provide useful information in the form of various ventilatory data regarding a physical condition of a patient and/or a prescribed respiratory treatment for the patient. The useful information may be derived by the ventilator 202, based on data received by a patient data module 210, collected by a monitor module 220, and compiled by a data processing module 222. The useful information may be displayed to the clinician in the form of graphs, wave representations, pie graphs, or other suitable forms of graphic display. For example, one or more prompts may be displayed on the GUI and/or user interface module 204 upon receipt of one or more proposed settings adjustments by the ventilator. Additionally or alternatively, the one or more prompts may be communicated to a remote monitoring system coupled via any suitable means to the ventilatory system 200.

### Equation of Motion

Ventilation module 214 may oversee ventilation of a patient according to ventilatory settings received via settings module 212. By way of general overview, the basic elements impacting ventilation may be described by the following ventilatory equation (also known as the Equation of Motion):

PₘPᵥ=V_{T}/C+R*F

Here, Pₘ is a measure of muscular effort that is equivalent to the pressure generated by the muscles of a patient. If the patient's muscles are inactive, the Pₘ is equivalent to 0 cm H₂O. During inspiration, Pᵥ represents the positive pressure delivered by a ventilator (generally in cm H₂O). V_{T} represents the tidal volume delivered based on the pressure supplied, C refers to the respiratory compliance, R represents the respiratory resistance, and F represents the gas flow during inspiration (generally in liters per min (L/m)). Alternatively, during exhalation, the Equation of Motion may be represented as:

Pₐ+Pₜ=V_{TE}/C+R*F

Here, Pₐ represents the positive pressure existing in the lungs (generally in cm H₂O), Pₜ represents the transairway pressure, V_{TE} represents the tidal volume exhaled, C refers to the respiratory compliance, R represents the respiratory resistance, and F represents the gas flow during exhalation (generally in liters per min (L/m)).

### Pressure

For positive pressure ventilation, pressure at the upper airway opening (e.g., in the patient's mouth) is positive relative to the pressure at the body's surface (i.e., relative to the ambient atmospheric pressure to which the patient's body surface is exposed, about 0 cm H₂O). As such, when Pᵥ is zero, i.e., no ventilatory pressure is being delivered, the upper airway opening pressure will be equal to the ambient pressure (i.e., about 0 cm H₂O). However, when inspiratory pressure is applied (i.e., positive pressure), a pressure gradient is created that allows gases to flow into the airway and ultimately into the lungs of a patient during inspiration (or, inhalation) until the pressure is equalized. When an inspiratory volume (or V_{T}) has been delivered to the lungs such that the inspiratory pressure is achieved and maintained, pressure is equalized and gases no longer flow into the lungs (i.e., zero flow).

### Flow and Volume

Volume refers to the amount of gas delivered to a patient's lungs, usually in liters (L). Flow refers to a rate of change in volume over time (F=ΔV/Δt). Flow is generally expressed in liters per minute (L/m or lpm) and, depending on whether gases are flowing into or out of the lungs, flow may be referred to as inspiratory flow or expiratory flow, respectively. According to embodiments, the ventilator may control the rate of delivery of gases to the patient, i.e., inspiratory flow, and may control the rate of release of gases from the patient, i.e., expiratory flow.

As may be appreciated, volume and flow are closely related. That is, where flow is known or regulated, volume may be derived based on elapsed time. Indeed, volume may be derived by integrating the flow waveform. According to embodiments, a tidal volume, V_{T}, may be delivered upon reaching a set inspiratory time (T_{I}) at set inspiratory flow. Alternatively, set V_{T} and set inspiratory flow may determine the amount of time required for inspiration, i.e., T_{I}.

### Respiratory Compliance

Additional ventilatory parameters that may be measured and/or derived may include respiratory compliance and respiratory resistance, which refer to the load against which the patient and/or the ventilator must work to deliver gases to the lungs. Respiratory compliance may be interchangeably referred to herein as compliance. Generally, compliance refers to a relative ease with which something distends and is the inverse of elastance, which refers to the tendency of something to return to its original form after being deformed. As related to ventilation, compliance refers to the lung volume achieved for a given amount of delivered pressure (C=ΔV/ΔP). Increased compliance may be detected when the ventilator measures an increased volume relative to the given amount of delivered pressure. Some lung diseases (e.g., acute respiratory distress syndrome (ARDS)) may decrease compliance and, thus, require increased pressure to inflate the lungs. Alternatively, other lung diseases may increase compliance, e.g., emphysema, and may require less pressure to inflate the lungs.

### Respiratory Resistance

Respiratory resistance refers to frictional forces that resist airflow, e.g., due to synthetic structures (e.g., endotracheal tube, expiratory valve, etc.), anatomical structures (e.g., bronchial tree, esophagus, etc.), or viscous tissues of the lungs and adjacent organs. Respiratory resistance may be interchangeably referred to herein as resistance. Resistance is highly dependant on the diameter of the airway. That is, a larger airway diameter entails less resistance and a higher concomitant flow. Alternatively, a smaller airway diameter entails higher resistance and a lower concomitant flow. In fact, decreasing the diameter of the airway results in an exponential increase in resistance (e.g., two-times reduction of diameter increases resistance by sixteen times). As may be appreciated, resistance may also increase due to a restriction of the airway that is the result of, inter alia, increased secretions, bronchial edema, mucous plugs, brochospasm, and/or kinking of the patient interface (e.g., invasive endotracheal or tracheostomy tubes). Resistance may be expressed in centimeters of water per liter per second (i.e., cm H₂O/L/s).

### I:E Ratio

According to embodiments, an I:E ratio may be calculated (for a triggering patient) or set (for a non-triggering patient). According to embodiments, a normal patient (e.g., with a normal T_{I} and a normal RR for the patient's PBW) may have an I:E ratio of 1:2 to 1:3. That is, under normal conditions, it may be desirable for the T_{E} to be double or triple (or even greater) the set T_{I}. However, under some circumstances the length of the T_{E} approaches T_{I}. For instance, if set RR is too high (for a non-triggering patient), T_{E} may be too short and may not allow for complete exhalation. Alternatively, when inspiratory pressure is too high (causing increased delivered volume), T_{E} may be too short and may not allow for complete exhalation of the increased delivered volume. As described previously, when T_{E} is too short, gas-trapping may occur at the end of exhalation causing Auto-PEEP.

### Patient Data

According to embodiments, patient data may be received by the patient data module 210. As described above, patient data (including a patient diagnosis, a patient disability, a patient post-operative condition, a patient body weight, a patient gender, a patient age, etc.) may affect the projected impact of a proposed setting adjustment. As such, according to some embodiments, the ventilator may take into consideration patient data when calculating the projected impact, impact level, and/or an associated alert upon receiving a proposed setting adjustment. The ventilator may further take into consideration patient data when determining one or more alternative settings adjustments.

Some patients may exhibit certain characteristics associated with various conditions and diseases, e.g., COPD, ARDS, post-operative condition (single lung, cardiac surgery), etc. For example, patients diagnosed with COPD may exhibit chronic elevated resistance due to constricted airways, while ARDS patients may exhibit chronic elevated resistance due to alveolar collapse. In some cases, patients diagnosed with various conditions and diseases associated with an obstructive component may exhibit elevated resistance over many months or years. According to some embodiments, patients having these conditions may also exhibit elevated compliance.

According to embodiments described herein, a clinician may input a patient diagnosis, e.g., COPD, ARDS, emphysema, etc. The ventilator may associate the patient diagnosis with certain lung and airway characteristics. For example, if the ventilator receives a patient diagnosis of COPD, the ventilator may associate this patient diagnosis with elevated resistance. The ventilator may further associate this patient diagnosis with an obstructive component. Alternatively, if the ventilator receives a patient diagnosis of emphysema, the ventilator may associate this patient diagnosis with elevated compliance. Alternatively still, a patient diagnosis of ARDS may be associated with reduced lung compliance.

By way of example, implications of Auto-PEEP may by impacted by a patient diagnosis associated with COPD. As will be described further herein, Auto-PEEP is a dangerous condition associated with gas-trapping in the lungs. According to embodiments, upon receiving a proposed setting adjustment, the ventilator may determine that the proposed adjustment may implicate Auto-PEEP for a normal patient, but may not implicate Auto-PEEP for an obstructed patient (or visa versa). As such, an Auto-PEEP alert may be issued with the prompt for the normal patient, but not for the obstructed patient (or visa versa). As such, the ventilator may be configured to evaluate a patient diagnosis, or other patient data, when determining the projected impact of a proposed setting adjustment.

By way of alternative example, pressure and/or volume control may be impacted by a patient diagnosis. For example, atelectasis and edema may reduce aerated lung volumes in patients with acute lung injury and the ARDS. As a result, an ARDS patient may exhibit reduced lung compliance and additional pressure must be provided to achieve a particular tidal volume (ΔV=C*ΔP). As such, the additional inspiratory pressure may be too high, suggesting the presence of excessive distention, or “stretch,” of the aerated lung. To prevent such lung distention, whereas a normal patient may have a target V_{T} of 10 mL/kg, an ARDS patient may have a target V_{T} of only 7 mL/kg. Thus, if a proposed setting adjustment was projected to increase V_{T} from 7 mL/kg to 10 mL/kg for an ARDS patient, the ventilator may issue an alert. In contrast, the same proposed setting adjustment for a normal patient may not generate an alert.

By way of further example, patient data may also include a patient's age. For a neonatal patient, the patient data may include a gestational age (literally, the age of the fetus or newborn from the mother's last menstrual period (LMP)). Among other things, the gestational age may be used to estimate a developmental stage of a newborn. As may be relevant here, oxygen saturation above a particular level may be harmful to retinal vessels of newborn with a younger gestational age, whereas the same oxygen saturation may not be harmful to a newborn with an older gestational age. As such, depending on the gestational age of a neonatal patient, a proposed setting adjustment that is predicted to result in higher oxygen saturation may generate an alert for one neonatal patient and not for another neonatal patient.

### Inspiration

Ventilation module 214 may further include an inspiration module 216 configured to deliver gases to the patient according to ventilatory settings received by settings module 212. Specifically, inspiration module 216 may correspond to the inspiratory module 104 or may be otherwise coupled to source(s) of pressurized gases (e.g., air, oxygen, and/or helium), and may deliver gases to the patient. Inspiration module 216 may be configured to provide ventilation according to various ventilatory modes, e.g., via volume-targeted, pressure-targeted, or via any other suitable mode of ventilation.

According to embodiments, the inspiration module 216 may provide ventilation via a form of volume ventilation. Volume ventilation refers to various forms of volume-targeted ventilation that regulate volume delivery to the patient. Different modes of volume ventilation are available depending on the specific implementation of volume regulation. For example, for volume-cycled ventilation, an end of inspiration is determined based on monitoring the volume delivered to the patient. According to embodiments, during volume ventilation, as volume and flow are regulated by the ventilator, delivered V_{T}, flow waveforms (or flow traces), and volume waveforms may be constant and may not be affected by variations in lung or airway characteristics (e.g., respiratory compliance and/or respiratory resistance). Alternatively, pressure readings may fluctuate based on lung or airway characteristics. According to some embodiments, the ventilator may control the inspiratory flow and then derive volume based on the inspiratory flow and elapsed time. For volume-cycled ventilation, when the derived volume is equal to the prescribed V_{T}, the ventilator may initiate exhalation.

According to alternative embodiments, the inspiration module 216 may provide ventilation via a form of pressure ventilation. Pressure-targeted modes of ventilation may be provided by regulating the pressure delivered to the patient in various ways. For example, during pressure-cycled ventilation, an end of inspiration is determined based on monitoring the pressure delivered to the patient. According to embodiments, during pressure ventilation, the ventilator may maintain the same pressure waveform at the mouth, P_{aw}, regardless of variations in lung or airway characteristics, e.g., respiratory compliance and/or respiratory resistance. However, the volume and flow waveforms may fluctuate based on lung and airway characteristics. As noted above, pressure delivered to the upper airway creates a pressure gradient that enables gases to flow into a patient's lungs. The pressure from which a ventilator initiates inspiration is termed the end-expiratory pressure (EEP) or “baseline” pressure. This pressure may be atmospheric pressure (about 0 cm H₂O), also referred to as zero end-expiratory pressure (ZEEP). However, commonly, the baseline pressure may be positive, termed positive end-expiratory pressure (PEEP). Among other things, PEEP may promote higher oxygenation saturation and/or may prevent alveolar collapse during exhalation. Under pressure-cycled ventilation, upon delivering the inspiratory pressure the ventilator may initiate exhalation.

According to still other embodiments, a combination of volume and pressure ventilation may be delivered to a patient, e.g., volume-targeted-pressure-controlled (VC+) ventilation. In particular, VC+ ventilation may provide benefits of setting a target. V_{T}, while also allowing for monitoring variations in flow. As will be detailed further below, variations in flow may be indicative of various patient conditions.

### Exhalation

Ventilation module 214 may further include an exhalation module 218 configured to release gases from the patient's lungs according to prescribed ventilatory settings. Specifically, exhalation module 218 may correspond to expiratory module 108 or may otherwise be associated with and/or controlling an expiratory valve for releasing gases from the patient. By way of general overview, a ventilator may initiate exhalation based on lapse of an inspiratory time setting (T_{I}) or other cycling criteria set by the clinician or derived from ventilator settings (e.g., detecting delivery of prescribed V_{T} or prescribed inspiratory pressure based on a reference trajectory). Upon initiating the expiratory phase, exhalation module 218 may allow the patient to exhale by opening an expiratory valve. As such, exhalation is passive, and the direction of airflow, as described above, is governed by the pressure gradient between the patient's lungs (higher pressure) and the ambient surface pressure (lower pressure). Although expiratory flow is passive, it may be regulated by the ventilator based on the size of the expiratory valve opening.

Expiratory time (T_{E}) is the time from the end of inspiration until the patient triggers for a spontaneously breathing patient. For a non-triggering patient, it is the time from the end of inspiration until the next inspiration based on the set RR. In some cases, however, the time required to return to the functional residual capacity (FRC) or resting capacity of the lungs is longer than provided by T_{E} (e.g., because the patient triggers prior to fully exhaling or the set RR is too high for a non-triggering patient). According to embodiments, various ventilatory settings may be adjusted to better match the time to reach FRC with the time available to reach FRC. For example, decreasing set T_{I} to thereby increase the amount of time available to reach FRC. Alternatively, inspiratory pressure may be decreased (decreasing V_{T}), resulting in less time required to reach FRC.

As may be further appreciated, at the point of transition between inspiration and exhalation, the direction of airflow may abruptly change from flowing into the lungs to flowing out of the lungs or vice versa depending on the transition. Stated another way, inspiratory flow may be measurable in the ventilatory circuit until P_{Peak} is reached, at which point flow approximates zero. Thereafter, upon initiation of exhalation, expiratory flow is measurable in the ventilatory circuit until the pressure gradient between the lungs and the body's surface reaches zero (again, resulting in zero flow). However, in some cases, as will be described further herein, expiratory flow may still be positive, i.e., measurable, at the end of exhalation (termed positive end-expiratory flow or positive EEF). In this case, positive EEF is an indication that the pressure gradient has not reached zero or, similarly, that the patient has not completely exhaled. Although a single occurrence of premature inspiration may not warrant concern, repeated detection of positive EEF may be indicative of Auto-PEEP.

### Ventilator Sensory Devices

The ventilatory system 200 may also include one or more distributed and/or internal sensors communicatively coupled to ventilator 202. Distributed sensors may communicate with various components of ventilator 202, e.g., ventilation module 214, internal sensors, monitor module 220, data processing module 222, and any other suitable components and/or modules. Distributed sensors may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors may further include pressure transducers that may detect changes in circuit pressure (e.g., electromechanical transducers including piezoelectric, variable capacitance, or strain gauge). Distributed sensors may further include various flow sensors for detecting airflow. For example, some flow sensors may use obstructions to create a pressure decrease corresponding to the flow across the device (e.g., differential pressure pneumotachometers) and other flow sensors may use turbines such that flow may be determined based on the rate of turbine rotation (e.g., turbine flow sensors). Alternatively, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Indeed, any distributed sensory device useful for monitoring changes in measurable parameters during ventilatory treatment may be employed in accordance with embodiments described herein.

Ventilator 202 may further include one or more internal sensors. Similar to distributed sensors, internal sensors may communicate with various components of ventilator 202, e.g., ventilation module 214, distributed sensors, monitor module 220, data processing module 222, and any other suitable components and/or modules. Internal sensors may employ any suitable sensory or derivative technique for monitoring one or more parameters associated with the ventilation of a patient. However, the one or more internal sensors may be placed in any suitable internal location, such as, within the ventilatory circuitry or within components or modules of ventilator 202. For example, sensors may be coupled to the inspiratory and/or expiratory modules for detecting changes in, for example, circuit pressure and/or flow. Specifically, internal sensors may include pressure transducers and flow sensors for measuring changes in circuit pressure and airflow. Additionally or alternatively, internal sensors may utilize optical or ultrasound techniques for measuring changes in ventilatory parameters. For example, a patient's expired gases may be monitored by internal sensors to detect physiological changes indicative of the patient's condition and/or treatment, for example. Indeed, internal sensors may employ any suitable mechanism for monitoring parameters of interest in accordance with embodiments described herein.

As should be appreciated, with reference to the Equation of Motion, ventilatory parameters are highly interrelated and, according to embodiments, may be either directly or indirectly monitored. For example, the distributed and internal sensors may provide raw data to the monitor module 220. The raw data may further be provided to the data processing module 222 for processing and deriving the collected ventilatory data. That is, parameters may be directly monitored by one or more sensors, as described above, or may be indirectly monitored by derivation according to the Equation of Motion.

### Collected Ventilatory Data

Ventilator 202 may further include a data processing module 222. As noted above, distributed sensors and internal sensors may collect data regarding various ventilatory parameters. A ventilatory parameter refers to any factor, characteristic, or measurement associated with the ventilation of a patient, whether monitored by the ventilator or by any other device. Sensors may further transmit collected data to the monitor module 220 and/or the data processing module 222. According to embodiments, the data processing module may 222 be configured to collect data regarding some ventilatory parameters, to derive data regarding other ventilatory parameters, and to graphically represent collected and derived data to the clinician and/or other modules of the ventilatory system. For example, data regarding end-expiratory flow (EEF), data regarding alveolar pressure Pₐ (e.g., via a breath-hold maneuver), P_{Peak} data, P_{Plat} data, volume data, flow trace data, EEP data, etc., may be collected, derived, and/or graphically represented by data processing module 222. Thereafter, the collected, derived, and/or graphically represented ventilatory data (hereinafter, the collected ventilatory data) may be utilized by the ventilator to calculate a projected impact of a proposed setting adjustment. Alternatively, the collected ventilatory data may be used to calculate one or more alternative settings adjustments.

### Flow Data

For example, according to embodiments, data processing module 222 may be configured to monitor inspiratory and expiratory flow. Flow may be measured by any appropriate, internal or distributed device or sensor within the ventilatory system. As described above, flow sensors may be employed by the ventilatory system to detect circuit flow. However, any suitable device either known or developed in the future may be used for detecting airflow in the ventilatory circuit. Data processing module 222 may be further configured to plot monitored flow data graphically via any suitable means. For example, according to embodiments, flow data may be plotted versus time (flow waveform), versus volume (flow-volume loop), or versus any other suitable parameter as may be useful to a clinician.

As may be appreciated, flow decreases as resistance increases, making it more difficult to pass gases into and out of the lungs (i.e., F=Pₜ/R). For example, when a patient is intubated, i.e., having either an endotracheal or a tracheostomy tube in place, resistance may be increased as a result of the smaller diameter of the tube over a patient's natural airway. In addition, increased resistance may be observed in patients with obstructive disorders, such as COPD, asthma, etc. Higher resistance may necessitate, inter alia, a higher inspiratory time setting (T_{I}) for delivering a prescribed pressure or volume of gases, a higher flow setting for delivering prescribed pressure or volume, a lower respiratory rate resulting in a higher expiratory time (T_{E}) for complete exhalation of gases, etc. According to embodiments, an evaluation of end-expiratory flow (EEF) may be used to detect Auto-PEEP, as described further herein. For example, if EEF has not reduced to zero before inspiration begins, this may indicate that gases may still be trapped in the lungs (e.g., insufficient T_{E} to return to FRC or elevated FRC).

### Pressure Data

According to embodiments, data processing module 222 may be configured to monitor pressure. Pressure may be measured by any appropriate, internal or distributed device or sensor within the ventilatory system. For example, pressure may be monitored by proximal electromechanical transducers connected near the airway opening (e.g., on the inspiratory limb, expiratory limb, at the patient interface, etc.). Alternatively, pressure may be monitored distally, at or near the lungs and/or diaphragm of the patient. Data processing module 222 may be further configured to plot monitored pressure data graphically via any suitable means. For example, according to embodiments, pressure data may be plotted versus time (pressure waveform), versus volume (pressure-volume loop or PV loop), or versus any other suitable parameter as may be useful to a clinician.

According to embodiments, PV loops may provide useful clinical and diagnostic information to clinicians regarding the respiratory resistance or compliance of a patient. Specifically, upon comparing PV loops from successive breaths, an increase in resistance may be detected when successive PV loops shorten and widen over time. That is, at constant pressure, less volume is delivered to the lungs when resistance is increasing, resulting in a shorter, wider PV loop. According to alternative embodiments, a PV loop may provide a visual representation, in the area between the inspiratory plot of pressure vs. volume and the expiratory plot of pressure vs. volume, which is indicative of respiratory compliance. Further, PV loops may be compared to one another to determine whether compliance has changed. Additionally or alternatively, optimal compliance may be determined. That is, optimal compliance may correspond to the dynamic compliance determined from a PV loop during a recruitment maneuver, for example.

### Volume Data

According to embodiments, data processing module 222 may be configured to derive volume via any suitable means. For example, as described above, during volume ventilation, a prescribed V_{T} may be set for delivery to the patient. The actual volume delivered may be derived by monitoring the inspiratory flow over time (i.e., V=F*T). Stated differently, integration of flow over time will yield volume. According to embodiments, V_{T} is completely delivered upon reaching T_{I}. Similarly, the expiratory flow may be monitored such that expired tidal volume (V_{TE}) may be derived. That is, under ordinary conditions, upon reaching the T_{E}, the prescribed V_{T} delivered should be completely exhaled and FRC should be reached. However, under some conditions T_{E} is inadequate for complete exhalation and FRC is not reached. Data processing module 222 may be further configured to plot derived volume data graphically via any suitable means. For example, according to embodiments, volume data may be plotted versus time (volume waveform), versus flow (flow-volume loop or FV loop), or versus any other suitable parameter as may be useful to a clinician.

### Determinations Regarding Proposed Settings Adjustments

According to embodiments, a proposed settings adjustments module 224 may evaluate, inter alia, collected ventilatory data, current ventilatory settings, and (optionally) patient data to determine a predicted impact of a proposed settings adjustment. For example, the predicted impact may include a projected impact (e.g., increasing P by x cm H₂O will increase V_{T} by about y mL/kg based on patient compliance, which V_{T} is relatively high for this patient having a particular PBW or diagnosed with a particular condition), an impact level (e.g., an intermediate impact level where resultant V_{T} is relatively high), and/or an alert (e.g., Auto-PEEP implicated based on collected ventilatory data).

### Projected Impact

As described above, according to embodiments, upon receipt of a proposed setting adjustment, a projected impact module 226 may determine a projected impact of the setting adjustment by any suitable means. For instance, the ventilator may calculate the projected impact based on, inter alia, current ventilatory settings, collected ventilatory data, patient data, and any suitable protocol, equation, etc. For example, the ventilator may first calculate a predicted result of the proposed setting adjustment using one or more suitable ventilatory equations, e.g., increasing P by x cm H₂O may be predicted to increase V_{T} by about y mL/kg based on patient compliance (note that according to alternative embodiments, a proposed increase in V_{T} may result in a corresponding predicted increase in P). Thus, according to this example, the predicted result may be an increase in V_{T} of about y mL/kg. Specifically, based on current ventilatory settings, the predicted result may increase V_{T} from about 7 mL/kg to about 9 mL/kg (i.e., y may be about 2 mL/kg). Thereafter, the ventilator may determine the projected impact of the predicted result on a particular ventilated patient. That is, the ventilator may evaluate current ventilatory settings, collected ventilatory data and/or patient data to determine the projected impact of the predicted result. For instance, in the case noted above, if the patient exhibits normal resistance and/or compliance, an increase in V_{T} from about 7 mL/kg to about 9 mL/kg may be determined to be within an acceptable range, e.g., by comparison with institutional protocol thresholds, manufacturer protocol thresholds, clinician protocol thresholds, prescribed patient ranges, etc. In contrast, if the patient has been diagnosed with ARDS, an increase in V_{T} from about 7 mL/kg to about 9 mL/kg may be determined to be outside an acceptable range and may be potentially harmful to the patient, e.g., by comparison with institutional protocol thresholds, manufacturer protocol thresholds, clinician protocol thresholds, prescribed patient ranges, etc. In this example, note that an ARDS patient would likely not exhibit the same compliance and, as such, a proposed increase in P by a different amount may have yielded this same predicted increase in V_{T}. According to embodiments, the prompt may display the projected impact in combination with the predicted result, e.g., increasing P by x cm H₂O will increase V_{T} by about 2 mL/kg, which may be harmful to this patient.

According to further embodiments, a projected impact may be calculated for a plurality of proposed settings adjustments. For instance, rather than making a single change to a ventilatory setting, a clinician may wish to make several changes to different ventilatory settings. In this case, one setting adjustment may offset another or one setting adjustment may compound another. As such, the ventilator may be configured to calculate a combined projected impact of the plurality of proposed settings adjustments. For example, in addition to the proposed setting adjustment described above (i.e., increasing P by x cm H₂O increases V_{T} by about 2 mL/kg based on patient compliance), the clinician may also wish to increase RR by 2 breaths/min. In this case, the ventilator may calculate one or more predicted results based on this additional proposed setting adjustment. For example, an increase in RR may decrease T_{I} by about x ms. In addition, based on the I:E ratio, an increase in RR may also decrease T_{E} by about y ins (where x and y may or may not be equivalent). Thereafter, for example, the ventilator may calculate the combined projected impact of the increase in V_{T} and the decrease T_{E}. Here, for a normal patient, whereas the increase in V_{T} alone may have been acceptable, the V_{T} increase in combination with decreased T_{E} may implicate Auto-PEEP (e.g., based on collected ventilatory data, protocols, equations, etc.). According to embodiments, the prompt may display the combined projected impact along with the combined predicted results (collectively termed the combined projected impact), e.g., increasing P by x cm H₂O will increase V_{T} by about 2 mL/kg, increasing RR by 2 breaths/min will decrease T_{E} by about y ms, these combined settings adjustments may cause Auto-PEEP.

### Impact Level

According to further embodiments, upon determination of a projected impact of a proposed setting adjustment, an impact level module 228 may determine a relative impact level of the proposed setting adjustment. An impact level may communicate, e.g., graphically or otherwise, the projected impact of a proposed setting adjustment for a particular patient. For example, the projected impact may be specified in a particular font (e.g., “which increase in V_{T} may be harmful to this ARDS patient” may be displayed in bold font to indicate a negative impact level). According to alternative embodiments, e.g., for an ARDS patient, the prompt may specify the predicted result (e.g., increasing P by x cm H₂O will increase V_{T} by about 2 mL/kg) and may communicate the projected impact graphically (e.g., a red-colored prompt to communicate a negative impact level or a prompt having an “exclamation mark” or “frowning face” icon to communicate a negative impact level). Alternatively, e.g., for a normal patient, the prompt issued in the previous example may display the predicted result (e.g., increasing P by x cm H₂O will increase V_{T} by about 2 mL/kg) on a green-colored prompt or on a prompt having a “smiling face” icon to communicate a positive impact level.

According to further embodiments, graphical impact levels may provide a means for a clinician to select an appropriate setting adjustment. For example, according to embodiments, a clinician may enter a proposed setting adjustment that may generate a yellow-colored prompt. The clinician may then scroll or toggle (e.g., via a toggle bar, scroll wheel, mouse, etc.) up and/or down from the proposed setting adjustment until a green-colored prompt is displayed. According to this embodiment, the clinician may select a setting adjustment based on a positive projected impact.

### Alerts

As described above, upon receipt of one or more proposed settings adjustments, an alert module 230 may determine that an adverse condition may be implicated for the patient, e.g., Auto-PEEP may be implicated. According to embodiments, in addition to an indication of an impact level (e.g., a yellow- or red-colored prompt or a prompt having an icon suggesting an intermediate or negative impact level), the ventilator may also issue an alert associated with the detected adverse condition.

For example, the ventilator may detect that one or more proposed settings adjustments may implicate and/or cause Auto-PEEP. As described above, in some cases, Auto-PEEP may result when the lungs are not sufficiently emptied during exhalation before inspiration is initiated. For example, gas-trapping may result when set RR is too high (for a non-triggering patient), when inspiration is initiated by the patient before exhalation is complete (for a triggering patient), when V_{T} is too high, T_{I} is too long and/or T_{E} is too short, etc. Specifically, when incomplete exhalation occurs, gases may be trapped in the lungs, resulting in an increased functional residual capacity (FRC). Indeed, with each breath, additional gases may be trapped and, not surprisingly, Auto-PEEP has been linked to barotrauma and an increase in the work of breathing (WOB), among other conditions.

Auto-PEEP may occur as a result of various patient conditions and/or inappropriate ventilatory settings. Thus, according to embodiments, the ventilator may evaluate various collected ventilatory data based on one or more predetermined thresholds to detect an implication of Auto-PEEP upon receiving a proposed setting adjustment. For example, the ventilator may evaluate expiratory flow on a flow waveform, or a flow trace, to determine whether EEF has reached zero before inspiration begins (i.e., if EEF is positive, the pressure gradient between the patient's lungs and the ambient surface pressure has likely not reached zero). According to further embodiments, the ventilator may evaluate collected ventilatory data to determine whether respiratory resistance is increasing (i.e., increased resistance may cause a decrease in flow and T_{E} may not be adequate for complete exhalation to FRC). According to further embodiments, the ventilator may evaluate various ventilatory parameters to determine whether respiratory compliance is increasing (i.e., higher V_{T} may be delivered at a constant pressure, requiring increased T_{E} to completely exhale). These examples are not comprehensive and other data may be evaluated to detect implications of Auto-PEEP (e.g., time to reach FRC vs. trended T_{E}, data from an expiratory-pause maneuver, etc.)

As described above, a proposed setting adjustment may increase the likelihood of Auto-PEEP. For example, a proposed setting adjustment that would increase RR (lowering T_{E}), increase V_{T} (increasing the time required to exhale), etc., may increase the likelihood of Auto-PEEP. Based on the collected ventilatory data, Auto-PEEP may be implicated by the proposed setting adjustment and the prompt may provide a corresponding alert (e.g., Auto-PEEP Implicated) in addition to the predicted result and/or projected impact (e.g., an indication of an intermediate impact level via a yellow-colored prompt or a suitable icon). Alternatively, based on the collected ventilatory data, the ventilator may determine that Auto-PEEP may be imminent upon acceptance of the proposed setting adjustment and the prompt may provide a corresponding alert (e.g., Auto-PEEP Imminent) in addition to the predicted result and/or projected impact (e.g., an indication of a red or negative impact level). According to some embodiments, in the case of a negative impact level, the ventilator may be configured to require an additional conformation from the clinician prior to acceptance of the proposed setting adjustment.

According to embodiments, an implication of Auto-PEEP is but one adverse condition that may be identified by a ventilator based on collected ventilatory data and/or patent data. According to embodiments, other appropriate alerts may be generated. For example, as described above, a proposed increase in the setting for delivered oxygen, which will likely increase arterial oxygen saturation, may be harmful to the retinal vessels of a neonatal patient of a particular gestational age. In this case, a corresponding alert may issue on the prompt (e.g., “Predicted Oxygen Saturation may be Harmful to Retinal Vessels”). By way of further example, as described above, a proposed increase in V_{T} above certain levels may be harmful to an ARDS patient. As such, a corresponding alert may issue on the prompt (e.g., “Predicted Increase in V_{T} Implicates Over-distension of Lungs”). Indeed, over-distension of ARDS patients' lungs has been directly linked to increased mortality (i.e., the prompt may also indicate a negative impact level).

The recited embodiments are not intended to be limiting and other suitable embodiments are well within the scope of the present disclosure.

### Alternative Ventilatory Settings Adjustments

Ventilator 202 may further include an alternative settings determination module 232. That is, according to embodiments, in addition to calculating a projected impact of one or more proposed settings adjustments, the ventilator may also calculate and recommend one or more alternative settings adjustments where appropriate. For instance, the ventilator may calculate one or more proposed settings adjustments based on, inter alia, current ventilatory settings, collected ventilatory data patient data, and any suitable protocol, equation, etc. For example, the ventilator may first identify a target ventilation change based on evaluating the proposed setting adjustment. That is, if the proposed setting adjustment increased V_{T} or RR, the ventilator may determine that the clinician seeks an increase in ventilation. Alternatively, if the proposed setting adjustment increased % O₂, the ventilator may determine that the clinician seeks an increase in oxygen saturation. According to alternative embodiments, the ventilator may present a dialog box or otherwise to the clinician to determine the clinician's intentions. Upon determining a target ventilation change (e.g., increasing ventilation, increasing oxygen saturation, etc.), the ventilator may determine one or more alternative settings adjustments to achieve the target ventilation change based on, inter alia, the patient diagnosis, the collected ventilatory data, the current ventilatory settings, and any suitable protocol, equation, etc.

According to embodiments, for example, suppose the proposed setting adjustment increased P by x cm H₂O, increasing V_{T} from about 7 mL/kg to about 9 mL/kg. Based on this proposed setting adjustment, the ventilator may determine that the clinician wishes to increase ventilation of the patient. However, further suppose that the patient is diagnosed with ARDS. As described above, an increase in V_{T} from about 7 mL/kg to about 9 mL/kg may cause over-distension of this patient's lungs. In this case, the ventilator may determine that increasing RR (where Auto-PEEP is not implicated) and/or increasing PEEP (where PEEP is not already high) may achieve increased ventilation without over-distending the lungs. As can be seen from this example, alternative settings adjustments may be evaluated based on achieving the target ventilatory change based on collected ventilatory data (e.g., indicating whether or not Auto-PEEP is a concern) and based on current ventilatory settings (e.g., indicating whether PEEP is already set high). According to embodiments, alternative settings adjustments may also be evaluated based on patient data (e.g., based on patient diagnosis, patient age, patient PBW, etc.). Indeed, as should be appreciated, any number of alternative settings adjustments may be determined based on a target ventilation change and the particular collected ventilatory data, current ventilatory settings, and patient data presented.

### Prompt Generation

Ventilator 202 may further include a prompt module 234. The prompt module 234 may provide a prompt regarding a projected impact of a proposed setting adjustment and/or recommendations for alternative settings adjustments via any suitable means. For example, the prompt may be provided as a tab, banner, dialog box, or other suitable type of display. Further, the prompt may be provided along a border of the graphical user interface, near an alarm display or bar, or in any other suitable location. A shape and size of the prompt may further be optimized for easy viewing with minimal interference to other ventilatory displays. The prompt may be further configured with a combination of icons and text such that the clinician may readily identify the predicted result and projected impact of a proposed setting adjustment. According to further embodiments, the prompt module 234 may provide an extended prompt via any suitable means. For example, the extended prompt may be selectably activated via any suitable means and may display the one or more alternative settings adjustments. The extended prompt may further be provided adjacent to the prompt (i.e., initial prompt) along a border of the graphical user interface, near an alarm display or bar, or in any other suitable location. The shape and size of the extended prompt may further be optimized for easy viewing with minimal interference to other ventilatory displays.

Prompt module 234 may also provide the prompt and/or extended prompt as a partially transparent window or format. The transparency may allow for the prompt and/or extended prompt to be displayed such that normal ventilator GUI and respiratory data may be visualized behind the prompts. This feature may be particularly useful for displaying the extended prompt. The prompts and/or extended prompts may further be displayed in areas of the user interface that are either blank or that cause minimal distraction from the respiratory data and other graphical representations provided by the GUI. However, upon selective expansion of a prompt, respiratory data and graphs may be at least partially obscured. As a result, prompt module 234 may provide the extended prompt such that it is partially transparent.

The prompt module 234 may further include a result/impact module 236. As described above, the impact of a proposed setting adjustment may be difficult for a clinician to estimate. As may be appreciated, collected ventilatory data, current ventilatory settings, and/or patient data may be evaluated by the ventilator in order to determine a projected impact and impact level of one or more proposed settings adjustments. Thereafter, the result/impact module 236 may be configured to provide a prompt to the clinician indicating, for example, a predicted result and a projected impact and/or impact level. As described above, the predicted result may refer to how a proposed setting adjustment may affect various ventilatory parameters that may not be readily apparent to a clinician. The projected impact and/or impact level may refer to how the predicted result may impact patient condition and/or patient treatment. For example, result/impact module 236 may be configured to display the prompt on user interface 204, e.g., within a window of the GUI. According to additional embodiments, the prompt may be communicated to and/or displayed on a remote monitoring system communicatively coupled to ventilatory system 200.

Specifically, the result/impact module 236 may provide a predicted result of the proposed setting adjustment on the prompt in text form (e.g., “increasing P by x cm H₂O will increase V_{T} by about y mL/kg based on a patient compliance”). The result/impact module 236 may also state a projected impact of the proposed setting adjustment in text form (e.g., “increasing V_{T} by about y mL/kg may adversely affect this patient”). Alternatively, the result/impact module 236 may communicate the projected impact by providing a color impact level indication on the prompt (e.g., “increasing P by x cm H₂O will increase V_{T} by about y mL/kg” displayed on a red-colored prompt to communicate a negative impact level). Alternatively, the result/impact module 236 may communicate the projected impact by providing an icon impact level indication on the prompt (e.g., “increasing P by x cm H₂O will increase V_{T} by about y mL/kg” displayed on a prompt having an “exclamation mark” or “frowning face” icon to communicate a negative impact level). Alternatively still, the result/impact module 236 may communicate the projected impact by providing a font impact level indication on the prompt (e.g., “increasing P by x cm H₂O will increase V_{T} by about y mL/kg” presented in bold font to communicate a negative impact level). As should be appreciated, the described embodiments are provided as examples only and any suitable method for presenting a predicted result and/or projected impact of a proposed setting adjustment may be employed within the spirit of the present disclosure.

The prompt module 234 may further include a recommend module 238. That is, the ventilator may also determine alternative settings adjustments upon receipt of a proposed setting adjustment. As described above, a target ventilation change may be determined based on the proposed setting adjustment. Further, one or more alternative settings adjustments may be calculated to achieve the target ventilation change based on, inter alia, current ventilatory settings, collected ventilatory data, patient data, and any suitable protocol, equation, etc. For example, recommend module 238 may be configured to display the alternative settings adjustment on the prompt on user interface 204, e.g., within a window of the GUI. According to additional embodiments, the prompt may be communicated to and/or displayed on a remote monitoring system communicatively coupled to ventilatory system 200.

Specifically, the recommend module 238 may provide recommendations for one or more alternative settings adjustments on the prompt in text form (e.g., where Auto-PEEP is implicated by the proposed setting adjustment, “consider decreasing V_{T} so that T_{E} may be adequate to completely exhale V_{T}”). The recommend module 238 may provide the one or more alternative settings adjustments on an initial prompt (e.g., with the predicted result and projected impact) or on an extended prompt. That is, according to embodiments, the initial prompt may provide an icon or other selectable control such that upon clinician selection the one or more alternative settings adjustments may be displayed. As should be appreciated, the described embodiments are provided as examples only and any suitable method for presenting a predicted result and/or projected impact of a proposed setting adjustment may be employed within the spirit of the present disclosure.

In order to accomplish the various aspects of the projected impact and/or recommendations display, the prompt module 234 may communicate with various other components and/or modules. For instance, prompt module 234 may be in communication with patient data module 210, settings module 212, monitor module 220, data processing module 222, proposed settings adjustments module 224, alternative settings determination module 232, or any other suitable module or component of the ventilatory system 200. That is, prompt module 234 may receive collected ventilatory data and information regarding the patient's ventilatory settings and treatment. Further, according to some embodiments, the prompt module 234 may have access to a patient's diagnostic information (e.g., regarding whether the patient has ARDS, COPD, asthma, emphysema, or any other disease, disorder, or condition).

According to embodiments, a prompt may issue automatically upon receipt of a proposed setting adjustment. Alternatively, a prompt may not issue unless and until a “review” or “test” button has been activated by a clinician. According to some embodiments, a “review” or “test” button may appear as a suggestion prior to a clinician accepting a proposed setting adjustment. According to alternative embodiments, a “review” or “test” button may be provided adjacent to an “accept” button on a settings adjustment screen of the GUI. According to further embodiments, a prompt may issue after each of a plurality of proposed settings adjustments are input (e.g., automatically or in response to activation of a “review” or other control). In this case, each successive prompt may provide a combined predicted result and combined projected impact based on each of the previous proposed settings adjustments. Alternatively or additionally, upon receipt of an indication that all of the plurality of proposed settings adjustments have been input or otherwise, the ventilator may issue a single combined prompt that identifies a combined predicted result and combined projected impact of all of the plurality of proposed settings adjustments. As should be appreciated, a number of suitable methods for issuing a prompt are possible and are well within the spirit of the present disclosure.

According to embodiments, upon viewing a prompt and/or extended prompt in response to receipt of a proposed setting adjustment, the prompt and/or extended prompt may be cleared from the graphical user interface.

### Methods of Prompt Generation

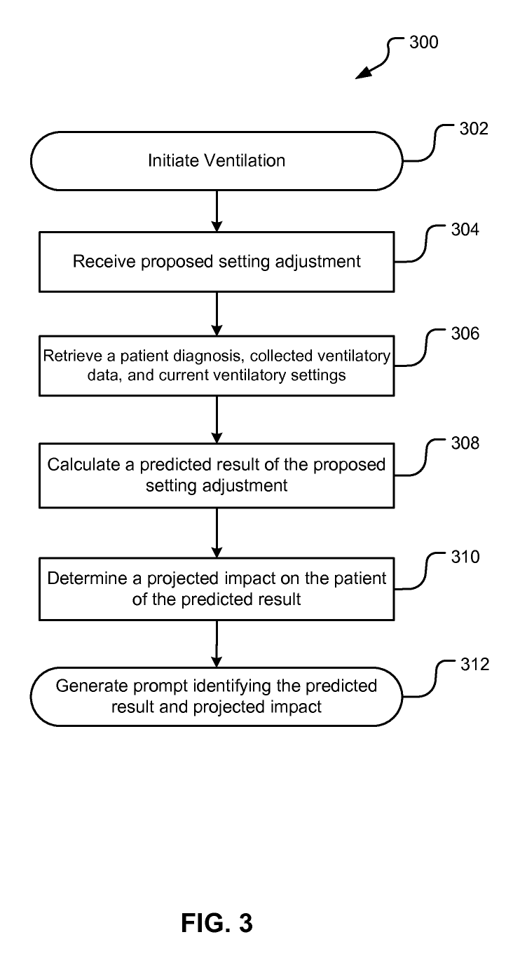

FIG. 3 is a flow chart illustrating an embodiment of a method for issuing a prompt identifying a projected impact of a proposed setting adjustment based on a patient diagnosis and collected ventilatory data.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

Method 300 begins with an initiate ventilation operation 302. Initiate ventilation operation 302 may further include various additional operations. For example, initiate ventilation operation 302 may include receiving one or more ventilatory settings associated with ventilation of a patient. As such, the ventilatory settings and/or input received may include, inter alia, an inspiratory pressure (or target inspiratory pressure), a tidal volume (V_{T}), a respiratory rate (RR), an I:E ratio, a % O₂, etc. Additionally, during ventilation, ventilatory settings may be adjusted and accepted (prior to the proposed settings adjustments referred to herein). These received ventilatory settings and previous ventilatory adjustments may hereinafter be referred to as current ventilatory settings (as differentiated from proposed settings adjustments). In addition, during initiate ventilation operation 302, patient data may be received. Patient data may refer to any data particular to a patient, for example, a predicted or ideal body weight (PBW or IBW), a patient diagnosis, a patient age, a patient disability, a patient post-operative condition, etc. A patient diagnosis may include, inter alia, ARDS, COPD, emphysema, asthma, etc. Upon initiating ventilation, the ventilator may further monitor ventilatory parameters and collect ventilatory data.

At receive operation 304, the ventilator may receive a proposed setting adjustment. The proposed setting adjustment may be received according to any suitable means. For example, a clinician may activate a settings screen on the user interface and may input a proposed setting adjustment via a keyboard, scroll wheel, touch screen, etc. The proposed setting adjustment may be received as a pending change such that the proposed setting adjustment may not become permanent unless and until accepted by the clinician. According to alternative embodiments, a plurality of proposed settings adjustments may be received at receive operation 304.

At retrieve operation 306, the ventilator may retrieve current ventilatory settings from the settings module, may retrieve patient data (including a patient diagnosis) from the patient data module, and may retrieve collected ventilatory data from the data processing module, for example. According to embodiments, the ventilator may further retrieve any other suitable data or information as needed for determining a projected impact of a proposed setting adjustment. According to embodiments, the ventilator may retrieve needed data and information from any suitable module of the ventilator or any other device, sensory apparatus, or monitoring apparatus communicatively coupled to the ventilator.

At calculate predicted result operation 308, the ventilator may calculate a predicted result of the proposed setting adjustment. The ventilator may calculate the predicted result based on, inter alia, current ventilatory settings, patient data, collected ventilatory data, and/or any other useful information in the form of equations, conversion tables, etc. For example, according to embodiments, the ventilator may calculate that increasing P by x cm H₂O increases V_{T} by y mL/kg based on patient compliance. Specifically, based on current ventilatory settings, the ventilator may determine that the predicted result will increase V_{T} from 7 mL/kg to 9 mL/kg (i.e., y may be 2 mL/kg). According alternative embodiments, the ventilator may calculate a plurality of predicted results for a proposed setting adjustment. For example, the ventilator may calculate that increasing RR by x breaths/min will decrease T_{I} by z ms and T_{E} by q ms (where z and q may or may not be equivalent). According to still further embodiments, the ventilator may calculate a plurality of predicted results of a plurality of proposed settings adjustments. For example, the ventilator may receive a proposed setting adjustment for increasing P by x cm H₂O and a proposed setting adjustment for increasing RR by x breaths/min. As such, according to embodiments, the ventilator may calculate a plurality of predicted results, e.g.: (1) an increase in V_{T} of y mL/kg, (2) a decrease in T_{I} of z ms, and (3) a decrease in T_{E} of q ms. The ventilator may further make any other suitable calculations as necessary, e.g., based on the current ventilatory settings, such as determining that an increase in V_{T} of y mL/kg increases V_{T} from 7 mL/kg to 9 mL/kg.

At determine projected impact operation 310, the ventilator may determine a projected impact of the calculated predicted result(s). For example, the ventilator may determine the projected impact based on, inter alia, current ventilatory settings, patient data, collected ventilator), data, and/or any other useful information in the form of equations, conversion tables, protocols, etc. For example, according to embodiments, the ventilator may determine the projected impact of the predicted result on a particular ventilated patient. That is, where the patient exhibits normal resistance and/or compliance and where the predicted result suggests an increase in V_{T} from about 7 mL/kg to about 9 mL/kg, the ventilator may determine that this increase in V_{T} is within an acceptable range (e.g., by comparison with institutional protocol thresholds, manufacturer protocol thresholds, clinician protocol thresholds, prescribed patient ranges, etc.) In contrast, where the patient has been diagnosed with ARDS and where the predicted result suggests the same increase in V_{T} from about 7 mL/kg to about 9 mL/kg, the ventilator may determine that this increase in V_{T} is outside an acceptable range and may be potentially harmful to the patient (e.g., by comparison with institutional protocol thresholds, manufacturer protocol thresholds, clinician protocol thresholds, prescribed patient ranges, etc.).

According to further embodiments, a combined projected impact may be calculated for a plurality of proposed settings adjustments. For example, a plurality of proposed settings adjustments may include, inter alia, an increase in P by x cm H₂O, which may increase V_{T} by 2 mL/kg based on patient compliance, and increasing RR by 2 breaths/min. As described above, based on the I:E ratio, an increase in RR may decrease T_{E} by q ms. According to embodiments, at determine operation 310, the ventilator may calculate the combined projected impact of the increase in V_{T} and the decrease T_{E}. Here, for a normal patient, whereas the increase in V_{T} alone may have been acceptable, the V_{T} increase in combination with decreased T_{E} may implicate Auto-PEEP (e.g., based on collected ventilatory data). According to embodiments, the ventilator may take into consideration any useful information, e.g., in the form of protocols, clinical studies, etc., in determining correlations between predicted result(s) and a projected impact of one or more proposed settings adjustments.

At generate prompt operation 312, the ventilator may generate a prompt identifying the predicted result and the projected impact of one or more proposed settings adjustments by any suitable means. For example, the ventilator may generate a prompt identifying the predicted result and the projected impact in text form. That is, referring to the examples above, the ventilator may generate a prompt displaying the text “increasing P by x cm H₂O will increase V_{T} from about 7 mL/kg to about 9 mL/kg, which may adversely affect this patient.”

According to embodiments, the prompt may be selectively activated upon entry of one or more proposed settings adjustments, e.g., by a “review” control. Alternatively, according to embodiments, the prompt may be generated automatically upon receipt of each of one or more proposed settings adjustments. According to further embodiments, e.g., in the case of a plurality of settings adjustments, the clinician may indicate that all proposed settings have been input via any suitable means. Thereafter, the prompt may be displayed by any suitable means in any suitable location on the ventilator or a remote monitor. For example, the prompt may be displayed as a tab, banner, dialog box, or other suitable type of display, along a border of the graphical user interface, near an alarm display or bar, or in any other suitable location. The prompt may further be displayed in areas of the user interface that are either blank or that cause minimal distraction from the respiratory data and other graphical representations provided by the GUI. The prompt may be provided in a transparent form, or otherwise, for minimizing distraction, and may be cleared upon clinician viewing.

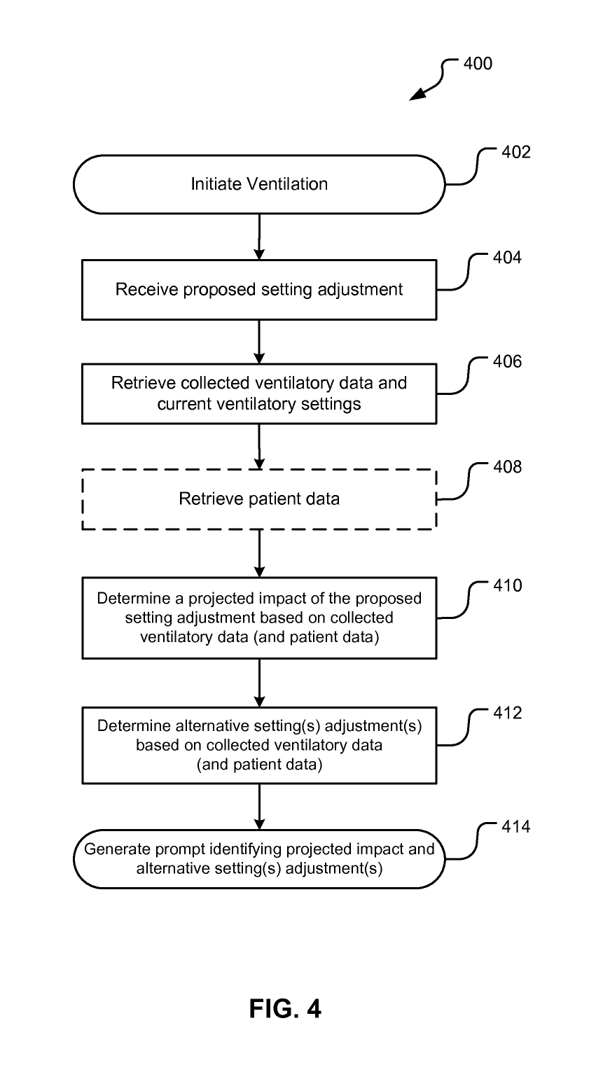

FIG. 4 is a flow chart illustrating an embodiment of a method for issuing a prompt identifying a projected impact of a proposed setting adjustment and suggesting one or more alternative settings adjustments.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

Method 400 begins with initiate ventilation operation 402, wherein the ventilator initiates ventilation, as described above in method 300.

At receive operation 404, the ventilator may receive a proposed setting adjustment, as described above with reference to operation 304.

At retrieve operation 406, the ventilator may retrieve current ventilatory settings from the settings module and may retrieve collected ventilatory data from the data processing module, for example, as described above with reference to operation 306.

At optional retrieve operation 408 (identified by dashed lines), the ventilator may optionally retrieve patient data (including a patient diagnosis) from the patient data module, as described above with reference to operation 306.

At determine projected impact operation 410, the ventilator may determine a projected impact of one or more proposed settings adjustments, as described above with reference to operations 308 and/or 310. In this case, the ventilator may or may not determine the projected impact based on patient data, e.g., a patient diagnosis. According to embodiments, a patient diagnosis may or may not be available to the ventilator for analysis. Additionally, a patient diagnosis may or may not be entered correctly by a clinician. If a diagnosis is not entered correctly, the ventilator may detect inconsistencies between the entered patient diagnosis and collected ventilatory data. In this case, according to embodiments, the ventilator may determine the projected impact based on collected ventilatory data and other available data and information.

At determine alternative settings adjustments operation 412, the ventilator may determine one or more alternative settings adjustments. For instance, the ventilator may determine one or more proposed settings adjustments based on, inter alia, the current ventilatory settings, collected ventilatory data, patient data, and/or any suitable protocol, equation, conversion chart, etc. For example, the ventilator may first identify a target ventilation change based on evaluating the one or more proposed settings adjustments and/or by querying the clinician. For example, the ventilator may determine that the clinician seeks an increase in ventilation. Upon determining a target ventilation change (e.g., increasing ventilation), the ventilator may determine one or more alternative settings adjustments to achieve the target ventilation change based on, inter alia, the current ventilatory settings, the collected ventilatory data, and optionally the patient data.

According to embodiments, referring to examples identified above, suppose the proposed setting adjustment increased P by x cm H₂O, increasing V_{T} from about 7 mL/kg to about 9 mL/kg. Based on this proposed setting adjustment, the ventilator may determine that the clinician wishes to increase ventilation of the patient. However, further suppose that the patient exhibits signs of Auto-PEEP. In this case, T_{E} may not be sufficient to completely exhale the increased V_{T} of about 2 mL/kg. As such, the ventilator may determine that decreasing RR in combination with the increased V_{T} may provide for increased ventilation without aggravating Auto-PEEP. That is, according to embodiments, the ventilator may recommend decreasing RR in addition to the proposed setting adjustment. Alternatively, with reference to the above example, the ventilator may determine that a reduced increase in V_{T}, e.g., of about 1 mL/kg, may increase ventilation without a need for decreasing RR. In this case, the ventilator may rather recommend an alternative setting adjustment of a reduced increase in P rather than an additional setting adjustment of a decrease in RR. As may be clear from the above example, various iterations of alternative settings adjustments may be appropriate under different circumstances, i.e., based on different collected ventilatory data, patient data, and/or any other suitable data or information (e.g., protocols, clinical studies, etc.).

At generate prompt operation 414, the ventilator may generate a prompt and/or an extended prompt identifying the projected impact of a proposed setting adjustment and one or more additional or alternative settings adjustments. According to embodiments, the ventilator may generate the prompt as described above with reference to operation 312. As such, the ventilator may provide the additional or alternative settings adjustments as recommendations on the prompt (or initial prompt). Alternatively, according to embodiments, the initial prompt may provide an icon or other selectable control such that upon clinician selection the one or more additional or alternative settings adjustments may be displayed on an extended prompt. The extended prompt may further be provided adjacent to the initial prompt along a border of the graphical user interface, near an alarm display or bar, or in any other suitable location. The shape and size of the extended prompt may further be optimized for easy viewing with minimal interference to other ventilatory displays. The initial prompt and/or extended prompt may be provided in a transparent form, or otherwise, for minimizing distraction, and may be cleared upon clinician viewing.

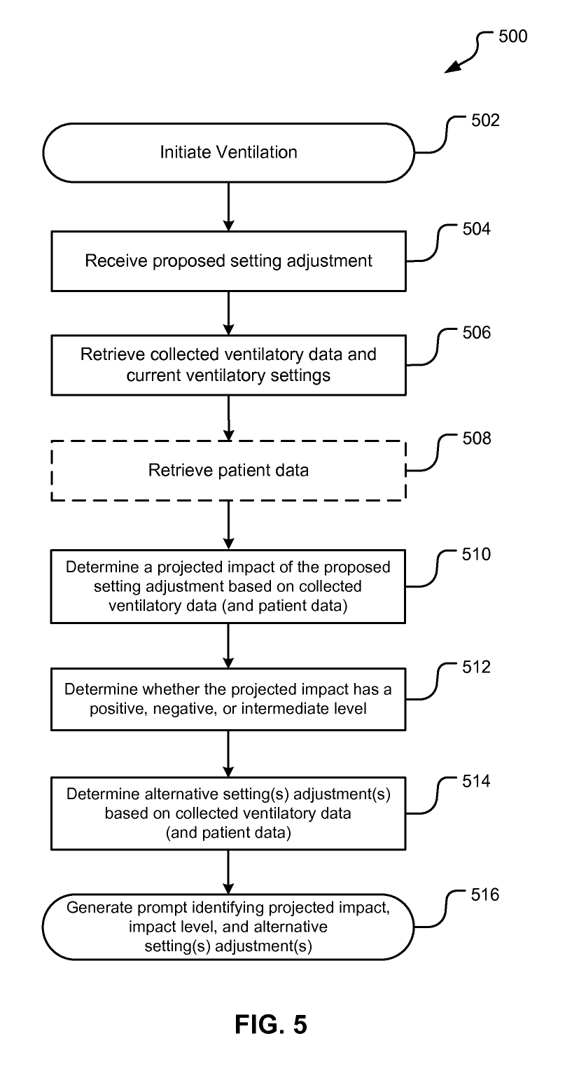

FIG. 5 is a flow chart illustrating an embodiment of a method for issuing a prompt identifying a projected impact and an impact level of a proposed setting adjustment and suggesting one or more alternative settings adjustments.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

Method 500 begins with initiate ventilation operation 502, wherein the ventilator initiates ventilation, as described above for initiate ventilation operation 302.

At receive operation 504, the ventilator may receive a proposed setting adjustment, as described above with reference to operation 304.

At retrieve operation 506, the ventilator may retrieve current ventilatory settings from the settings module and may retrieve collected ventilatory data from the data processing module, for example, as described above with reference to operation 306.

At optional retrieve operation 508 (identified by dashed lines), the ventilator may optionally retrieve patient data (including a patient diagnosis) from the patient data module, as described above with reference to operation 306.

At determine projected impact operation 510, the ventilator may determine a projected impact of one or more proposed settings adjustments, as described above with reference to operation 410.

At projected impact level 512, the ventilator may determine whether the projected impact is of a positive, negative, or intermediate level. That is, as described above, upon determining a projected impact, the ventilator may determine a relative impact level of the projected impact. For example, if a projected impact is determined to promote the ventilatory treatment of the patient based on prescribed settings, protocols, or otherwise, the ventilator may determine that a proposed setting adjustment has a positive impact level. Additionally, the ventilator may determine that the proposed setting adjustment promotes a stable patient condition (e.g., does not implicate Auto-PEEP or other adverse patient condition). In this ease, the ventilator may also determine that the proposed setting adjustment has a positive impact level. Alternatively, if the ventilator determines that the proposed setting adjustment may adversely affect the patient's condition and/or ventilatory treatment (e.g., imminently cause Auto-PEEP, likely cause over-distension of the lungs, etc.), the ventilator may determine that the proposed setting adjustment has a negative impact level. Alternatively still, the ventilator may determine that a proposed setting adjustment exhibits some increased probability of adversely affecting patient condition (e.g., Auto-PEEP is implicated, but not imminent). In this case, the ventilator may determine that the proposed setting adjustment has an intermediate impact level.

At determine alternative settings adjustments operation 514, the ventilator may determine one or more alternative settings adjustments, as described above with reference to operation 412.

At generate prompt operation 516, the ventilator may generate a prompt and/or an extended prompt identifying the projected impact of a proposed setting adjustment and one or more additional or alternative settings adjustments, as described above with reference to operations 414 and/or 312. Additionally, the ventilator may display the impact level of the proposed setting adjustment on the prompt. For example, as described above, an impact level may communicate, e.g., graphically or otherwise, the projected impact of a proposed setting adjustment for a particular patient. For example, the projected impact may be specified in a particular font to communicate the impact level of the proposed setting adjustment (e.g., “which increase in V_{T} may be harmful to this ARDS patient” may be displayed in bold font and/or italic font to indicate a negative impact level). Alternatively or additionally, the projected impact may be specified graphically to communicate the impact level of the proposed setting adjustment (e.g., “which increase in V_{T} may be harmful to this ARDS patient” may be displayed on a red-colored prompt or on a prompt having an “exclamation mark” or “frowning face” icon to indicate a negative impact level).

According to further embodiments, graphical impact levels may provide a means for a clinician to select an appropriate setting adjustment. For example, according to embodiments, a clinician may enter a proposed setting adjustment that may generate a yellow-colored prompt. The clinician may then scroll or toggle (e.g., via a toggle bar, scroll wheel, mouse, etc.) up and/or down from the proposed setting adjustment until a green-colored prompt is displayed. According to this embodiment, the clinician may select a setting adjustment based on a positive projected impact.

### Ventilator GUI Display of Prompt

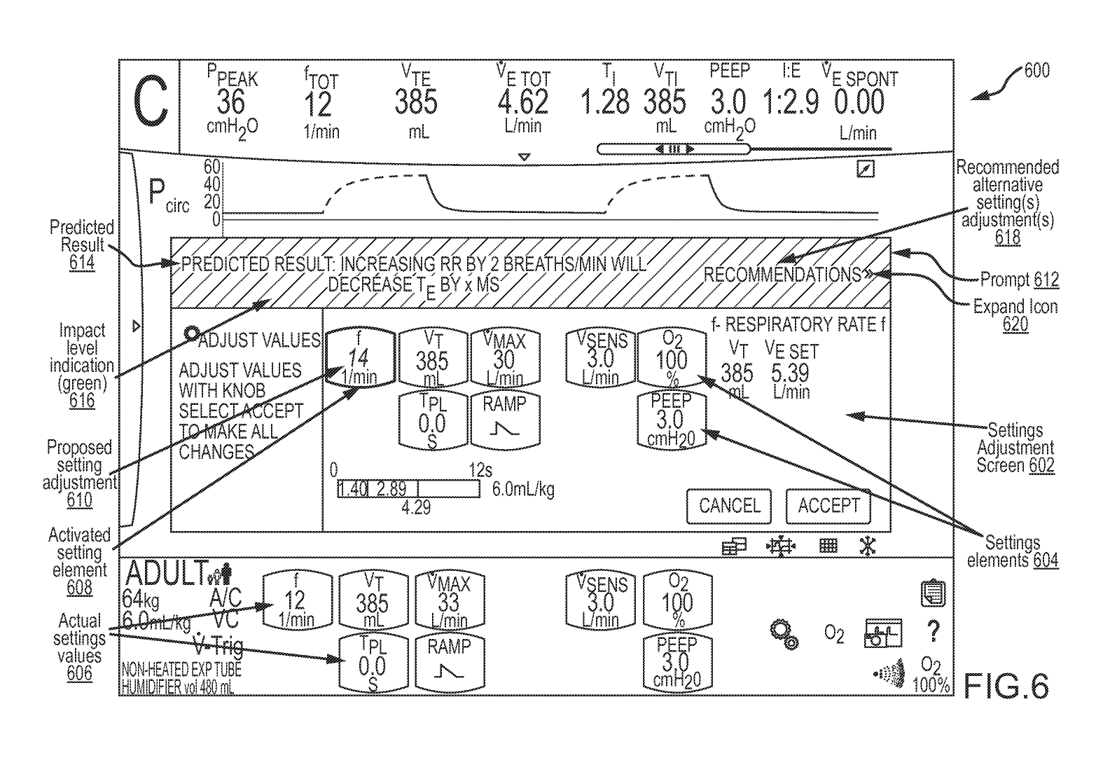

FIG. 6 is an illustration of an embodiment of a graphical user interface displaying a prompt identifying a projected impact and a positive impact level of a proposed setting adjustment.

Graphical user interface 600 may display a settings adjustment screen 602. The settings adjustment screen 602 may be accessed via any suitable means, e.g., via a settings icon or other control. The settings adjustment screen 602 may provide one or more settings elements 604. The one or more settings elements 604 may display actual settings values corresponding to current ventilatory settings, as displayed in actual settings values 606, for instance. A clinician may select an individual setting element, for example activated setting element 608, for adjustment. The activated setting element 608 may be identified by creation of a visual indication of selection, highlighting for example, such that it may be differentiated from unselected settings elements, e.g., settings elements 604.

Upon selection of a settings element, e.g., activated setting element 608, the clinician may input a proposed settings adjustment to a ventilatory parameter, e.g., proposed setting adjustment 610. Settings may be adjusted via any suitable means, for instance, via direct input into a settings input field, via use of a scroll wheel, thumbwheel, knob, mouse, or scroll bar for adjusting settings up and down, or via any other suitable device. According to embodiments, although a proposed setting adjustment may be input, the actual setting value associated with the ventilatory parameter may remain unchanged until the proposed setting value is accepted. For example, the current frequency (also referred to as RR) setting value of 12 breaths/min is represented in the actual settings values 606, while the proposed frequency value of 14 breaths/min is represented as proposed setting adjustment 610. According to embodiments, actual settings values may be represented in one font color and/or type (e.g., white font color and/or normal font type) and proposed settings values may be represented in another font color and/or type (e.g., yellow font color and/or italic font type). In the alternative, a proposed setting value may be represented in any suitable form such that the clinician may recognize that the setting value has a pending status. For instance, the proposed setting value may be displayed with an asterisk, or other indication.

Upon entry or input of proposed setting adjustment 610, a prompt may be displayed on the GUI, e.g., prompt 612. As described above, prompt 612 may be displayed as a tab, banner, dialog box, or other suitable type of display, along a border of the graphical user interface, near an alarm display or bar, or in any other suitable location. As illustrated, prompt 612 is displayed as a bar above and adjacent to the settings adjustment screen 602. However, prompt 612 may be located along any border region of the graphical user interface 600 (e.g., top, bottom, or side borders) (not shown) or in any other suitable location. Further, as described herein, prompt 612 may be partially transparent (not shown) such that ventilatory displays and data may be at least partially visible behind the prompt.

Prompt 612 may further provide a predicted result of proposed setting adjustment 610. As illustrated, proposed setting adjustment 610 indicates an increase in RR of 2 breaths/min (from 12 breaths/min to 14 breaths/min). As described above, the ventilator may calculate one or more predicted results of a proposed setting adjustment. For example, an increase in RR may decrease T_{I} and/or T_{E}. Based on current ventilatory settings (e.g., T_{I}, T_{E}, and I:E ratio), the ventilator may determine a predicted reduction in T_{I} and/or T_{E} (e.g., in ms) based on an increase in RR of 2 breaths/min. Prompt 612 may further display one or more predicted results of proposed setting adjustment 610, e.g., predicted result 614. In the illustrated embodiment, predicted result 614 states that “Increasing RR by 2 breaths/min will decrease T_{E} by x ms.” Predicted result 614 is provided by way of example only and “x ms” would be replaced by a calculated value in milliseconds during actual ventilation of a patient. According to alternative embodiments, a predicted reduction in T_{I} and/or resultant changes in other ventilatory parameters may be calculated and specified.

Prompt 612 may further provide an impact level indication, e.g., impact level indication 616. As described above, a prompt may identify whether a projected impact of a proposed setting adjustment is of a positive, negative, or intermediate level. As illustrated, the prompt is provided in a green color. According to embodiments, a green-colored prompt may indicate that the proposed setting adjustment has a positive impact level. According to additional or alternative embodiments, the prompt may display a “smiling face” icon (not shown) to communicate a positive impact level. That is, the proposed setting adjustment may promote a patient's ventilatory treatment and/or a stable patient condition, as described above.

Prompt 612 may further provide recommendations for one or more alternative settings adjustments, e.g., recommendations 618. As described above, the one or more additional or alternative settings adjustments may be provided on an initial prompt, e.g., prompt 612 (not shown). According to alternative embodiments, the one or more additional and/or alternative settings adjustments may be provided on an extended prompt (not shown). According to embodiments, an icon or other selectable control may be provided on the initial prompt for accessing the one or more additional or alternative settings adjustments. For example, expand icon 620 may be provided such that if a clinician desires to view recommendations for one or more additional or alternative settings adjustments, the expand icon 620 may be activated and the recommendations may be provided on an expanded prompt (not shown).

As may be appreciated, the disclosed data, graphics, and prompt illustrated in graphical user interface 600 may be arranged in any suitable order or configuration such that information and recommendations may be communicated to the clinician in an efficient and orderly manner. The disclosed data, graphics, and prompt are not to be understood as an exclusive array, as any number of similar suitable elements may be displayed for the clinician within the spirit of the present disclosure. Further, the disclosed data, graphics, and prompt are not to be understood as a necessary array, as any number of the disclosed elements may be appropriately replaced by other suitable elements without departing from the spirit of the present disclosure. The illustrated embodiment of the graphical user interface 600 is provided as an example only, including potentially useful information and recommendations that may be provided to the clinician to facilitate communication of a projected impact of a proposed settings adjustment in an orderly and informative way, as described herein.

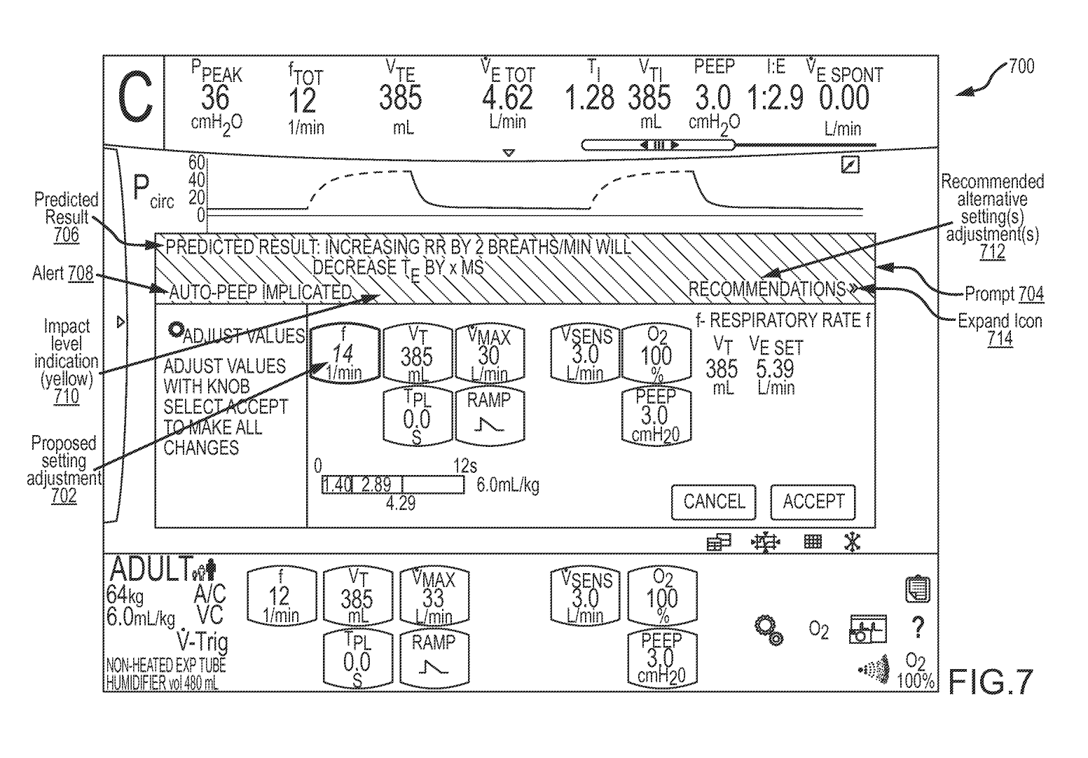

FIG. 7 is an illustration of an embodiment of a graphical user interface displaying a prompt identifying a projected impact having an alert and an intermediate impact level of a proposed setting adjustment.

Graphical user interface 700 may display a settings adjustment screen that may provide one or more settings elements, as described with reference to graphical user interface 600. Upon selection of a settings element, the clinician may input a proposed settings adjustment to a ventilatory parameter, e.g., proposed setting adjustment 702. As described above, proposed setting adjustment 702 indicates an increase in RR of 2 breaths/min (from 12 breaths/min to 14 breaths/min).

As described above, upon entry or input of proposed setting adjustment 702, a prompt may be displayed on the GUI, e.g., prompt 704. Prompt 704 may further provide one or more predicted results of proposed setting adjustment 702, e.g., predicted result 706. In the illustrated embodiment, predicted result 706 states that “Increasing RR by 2 breaths/min will decrease T_{E} by x ms.”

Prompt 704 may further provide an alert associated with proposed setting adjustment 702. According to embodiments, an alert may communicate a predicted adverse result and/or patient condition that may be of interest to a clinician. For example, if Auto-PEEP is implicated by a proposed setting adjustment, the alert may be provided on the prompt along with an intermediate impact level identification (e.g., yellow-colored prompt and/or prompt having a “straight-faced” icon). Alternatively, if Auto-PEEP is determined to be an imminent result of a proposed setting adjustment, the alert may be provided on the prompt along with a negative impact level identification (e.g., red-colored prompt and/or prompt having an “exclamation mark” or “frowning face” icon).

As noted above, an increase in RR of 2 breaths/min was not predicted to have an adverse affect on the patient associated with graphical user interface 600. However, based on collected ventilatory data, patient data, etc., the same increase in RR of 2 breaths/min may be predicted to increase the likelihood of an adverse condition for a patient associated with graphical user interface 700. Thus, the illustrated embodiment of graphical user interface 700 provides alert 708, “Auto-PEEP Implicated,” on a yellow-colored prompt.

According to alternative embodiments, an alert may issue when a proposed increase in oxygen saturation may be harmful to the retinal vessels of a neonatal patient of particular gestational age. In this case, a corresponding alert may issue on the prompt such as “Predicted Oxygen Saturation may be Harmful to Retinal Vessels” (not shown). According to alternative embodiments, an alert may issue when a proposed increase in V_{T} above certain levels may be harmful to an ARDS patient. In that case, a corresponding alert may issue on the prompt such as “Predicted Increase in V_{T} Implicates Over-distension of Lungs” (not shown). Indeed, over-distension of the lungs of ARDS patients has been directly linked to increased mortality. Thus, the prompt may also indicate a negative impact level. As should be appreciated, suitable alerts may issue in any case in which a predicted result may adversely affect the patient in a particular way that may be of interest to a clinician. In this way, the prompt may provide clarification regarding why a particular proposed setting adjustment is predicted to result in an intermediate or negative impact level, as described further below.

As described above, prompt 704 may further provide an impact level indication, e.g., impact level indication 710. As described above, a prompt may identify whether a projected impact of a proposed setting adjustment is of a positive, negative, or intermediate level. As illustrated, the impact level indication 710 is a yellow-colored prompt. According to embodiments, a yellow-colored prompt may indicate that the proposed setting adjustment has an intermediate impact level. That is, the proposed setting adjustment may exhibit some increased probability of adversely affecting patient condition (e.g., Auto-PEEP is implicated, but not imminent). According to additional or alternative embodiments, the prompt may communicate an intermediate impact level via an icon (e.g., “straight-faced” icon) (not shown). In the illustrated embodiment, alert 708 may be provided for communicating why the proposed setting adjustment was leveled as intermediate (i.e., “Auto-PEEP Implicated”).

Prompt 704 may further provide recommendations for one or more alternative settings adjustments, e.g., recommendations 712, as described above with reference to recommendations 618. According to embodiments, expand icon 714 may be provided such that if a clinician desires to view the recommendations for one or more additional or alternative settings adjustments, the expand icon 714 may be activated and the recommendations may be provided on an expanded prompt (not shown).

As may be appreciated, the disclosed data, graphics, and prompt illustrated in graphical user interface 700 may be arranged in any suitable order or configuration such that information and recommendations may be communicated to the clinician in an efficient and orderly manner. The disclosed data, graphics, and prompt are not to be understood as an exclusive array, as any number of similar suitable elements may be displayed for the clinician within the spirit of the present disclosure. Further, the disclosed data, graphics, and prompt are not to be understood as a necessary array, as any number of the disclosed elements may be appropriately replaced by other suitable elements without departing from the spirit of the present disclosure. The illustrated embodiment of the graphical user interface 700 is provided as an example only, including potentially useful information and recommendations that may be provided to the clinician to facilitate communication of a projected impact of a proposed settings adjustment in an orderly and informative way, as described herein.

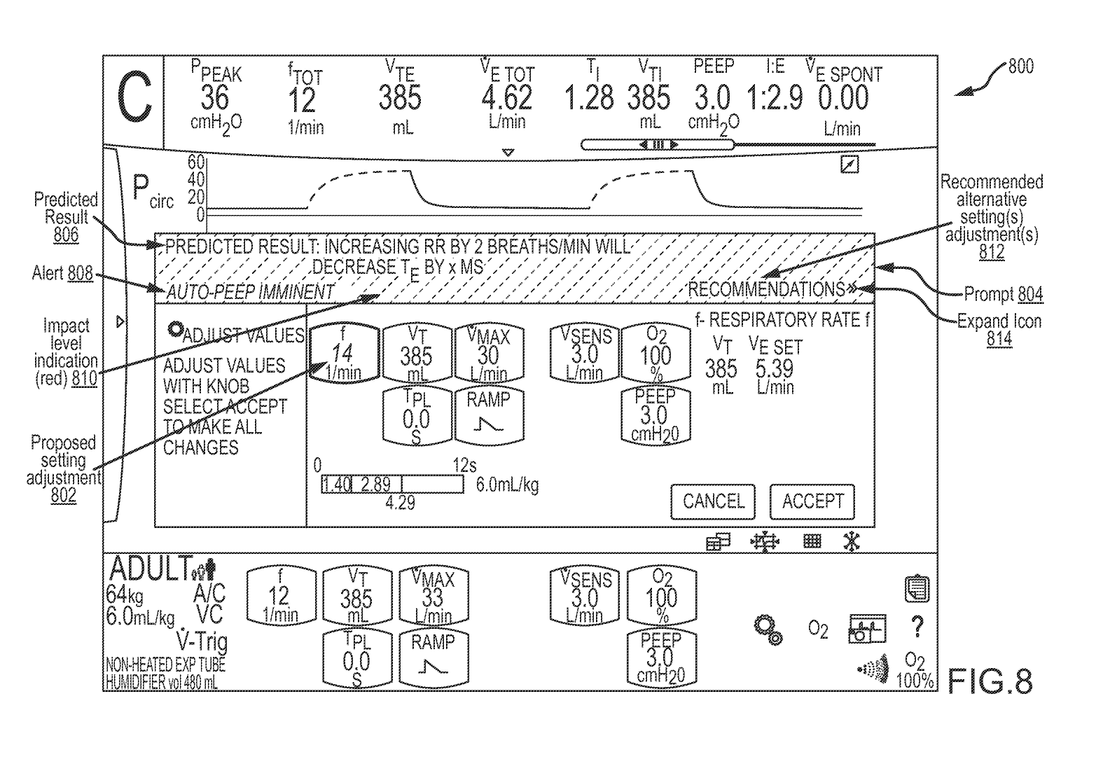

FIG. 8 is an illustration of an embodiment of a graphical user interface displaying a prompt identifying a projected impact having an alert and a negative impact level of a proposed setting adjustment.

Graphical user interface 800 may display a settings adjustment screen that may provide one or more settings elements, as described with reference to graphical user interfaces 600 and 700. Upon selection of a settings element, the clinician may input a proposed settings adjustment to a ventilatory parameter, e.g., proposed setting adjustment 802. As described above, proposed setting adjustment 802 indicates an increase in RR of 2 breaths/min (from 12 breaths/min to 14 breaths/min).

As described above, upon entry or input of proposed setting adjustment 802, a prompt may be displayed on the GUI, e.g., prompt 804. Prompt 804 may further provide one or more predicted results of proposed setting adjustment 802, e.g., predicted result 806. In the illustrated embodiment, predicted result 806 states that “Increasing RR by 2 breaths/min will decrease T_{E} by x ms.”

As with prompt 704, prompt 804 may further provide an alert associated with proposed setting adjustment 802. As noted above, an increase in RR of 2 breaths/min was predicted to increase the likelihood of an adverse condition for the patient associated with graphical user interface 700. However, for the same proposed setting adjustment, based on collected ventilatory data, patient data, etc., Auto-PEEP may be determined to be an imminent result for the patient associated with graphical user interface 800. In this case, alert 808 provides “Auto-PEEP Imminent” in bold, italic font.

According to alternative embodiments, an alert may issue when a proposed increase in oxygen saturation is predicted to be in a range that will likely harm the retinal vessels of a neonatal patient of particular gestational age. In this case, a corresponding alert may issue on the prompt such as “Predicted Oxygen Saturation Likely Harmful to Retinal Vessels.” According to alternative embodiments, an alert may issue when a proposed increase in V_{T} is predicted to be in a range that will likely injure the lungs of an ARDS patient. In that case, a corresponding alert may issue on the prompt such as “Predicted Increase in V_{T} Likely to Over-distend Lungs.” As should be appreciated, suitable alerts may issue in any case in which a predicted result is likely to adversely affect the patient in a particular way that may be of interest to a clinician. In this way, the prompt may provide clarification regarding why a proposed setting adjustment is predicted to result in a negative impact level, as described further below.

As described above, prompt 804 may further provide an impact level indication, e.g., impact level indication 810. As illustrated, the impact level indication 810 is a red-colored prompt. According to embodiments, a red-colored prompt may indicate that the proposed setting adjustment has a negative impact level. According to additional or alternative embodiments, a negative impact level may be communicated via an icon on the prompt (e.g., an “exclamation mark” or a “frowning face” icon) (not shown). That is, the proposed setting adjustment is likely to adversely affect patient condition and/or treatment. In the illustrated embodiment, alert 808 may be provided to communicate why the proposed setting adjustment was leveled as negative (i.e., “Auto-PEEP Imminent”).

Prompt 804 may further provide recommendations for one or more alternative settings adjustments, e.g., recommendations 812, as described above with reference to recommendations 712. According to embodiments, expand icon 814 may be provided such that if a clinician desires to view recommendations for one or more additional or alternative settings adjustments, the expand icon 814 may be activated and the recommendations may be provided on an expanded prompt (not shown).

As may be appreciated, the disclosed data, graphics, and prompt illustrated in graphical user interface 800 may be arranged in any suitable order or configuration such that information and recommendations may be communicated to the clinician in an efficient and orderly manner. The disclosed data, graphics, and prompt are not to be understood as an exclusive array, as any number of similar suitable elements may be displayed for the clinician within the spirit of the present disclosure. Further, the disclosed data, graphics, and prompt are not to be understood as a necessary array, as any number of the disclosed elements may be appropriately replaced by other suitable elements without departing from the spirit of the present disclosure. The illustrated embodiment of the graphical user interface 800 is provided as an example only, including potentially useful information and recommendations that may be provided to the clinician to facilitate communication of a projected impact of a proposed settings adjustment in an orderly and informative way, as described herein.

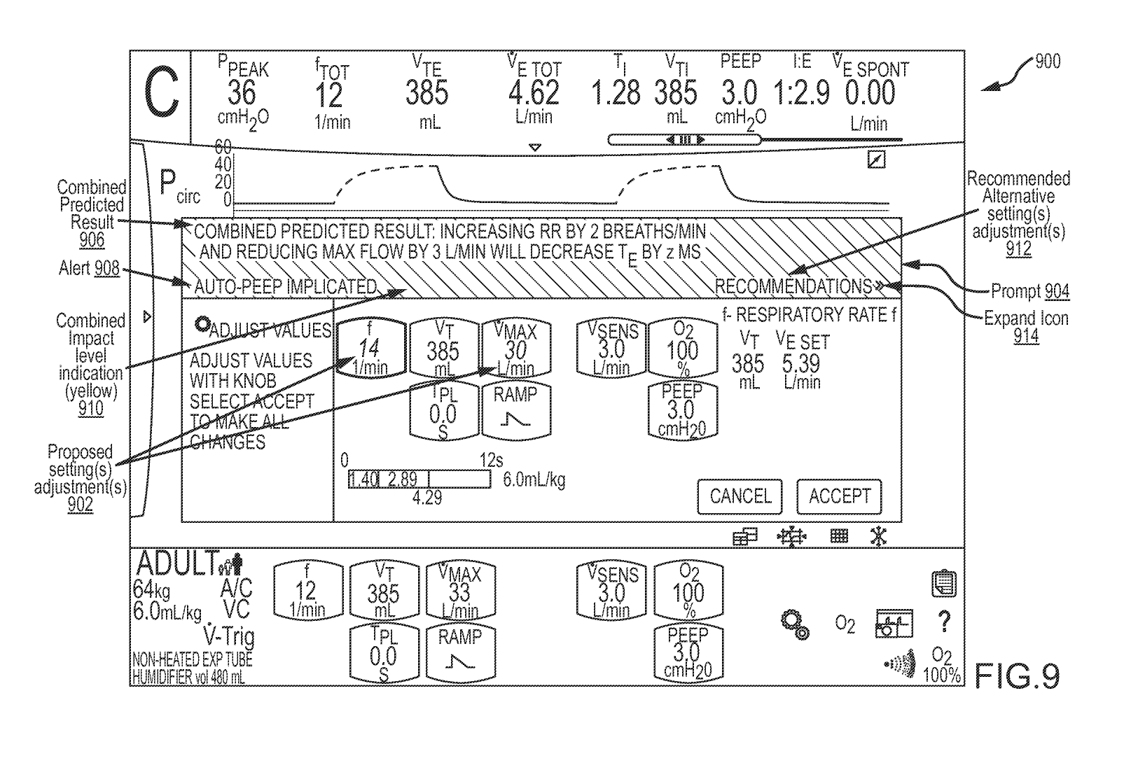

FIG. 9 is an illustration of an embodiment of a graphical user interface displaying a prompt identifying a projected impact having an alert and an intermediate impact level of a plurality of proposed settings adjustments.

Graphical user interface 900 may display a settings adjustment screen that may provide one or more settings elements, as described above. Upon selection of a plurality of settings elements, the clinician may input a plurality of proposed settings adjustments, e.g., proposed settings adjustments 902. In the illustrated embodiment, a first proposed setting adjustment of the plurality of proposed settings adjustments 902 involves decreasing max flow by 3 L/min, i.e., from 33 L/min to 30 L/min, A second proposed setting adjustment of the plurality of proposed settings adjustments 902 involves an increase in RR of 2 breaths/min (from 12 breaths/min to 14 breaths/min).

As described above, upon entry or input of the plurality of proposed settings adjustments 902, a prompt may be displayed on the GUI, e.g., prompt 904. Prompt 904 may further provide a combined predicted result of the plurality of proposed settings adjustments 902, e.g., combined predicted result 906. In this case, the first proposed setting adjustment, i.e., decreasing max flow by 3 L/min, may be predicted to increase T_{I} by x ms. That is, reducing max flow may increase the time required to deliver V_{T} (as F=ΔV/Δt). Further, at a constant RR, an increase in T_{I} may decrease T_{E} (i.e., an increase in T_{I} provides less time during each breath for T_{E} at constant RR). As such, the first proposed settings adjustment may be predicted to reduce T_{E} by y ms. Additionally, the second proposed setting adjustment, i.e., increasing RR by 2 breaths/min, may also reduce T_{E}. Specifically, an increase in the number of breaths per minute decreases the amount of time available for T_{I} and T_{E} associated with each breath. Thus, the combined predicted result of the first and second proposed settings changes may be predicted to reduce T_{E} by z ms. As such, according to the illustrated embodiment, combined predicted result 906 states “Increasing RR by 2 breaths/min and reducing max flow by 3 L/min will decrease T_{E} by z ms.”

According to further embodiments, a combined projected impact may be calculated for a plurality of proposed settings adjustments. For example, the ventilator may determine that a decrease in T_{E} by z ms may not provide sufficient time to exhale V_{T}. As such, the ventilator may determine that Auto-PEEP is implicated by the combined predicted result of the first and second proposed settings adjustments based on collected ventilatory data, patient data, etc. As such, the ventilator may issue alert 908, e.g., “Auto-PEEP Implicated.” Further, the ventilator may provide prompt 904 in a yellow color to communicate an intermediate impact level of the first and second proposed settings adjustments, i.e., combined impact level indication 910.

Prompt 904 may further provide recommendations for one or more alternative settings adjustments, e.g., recommendations 912, as described above with reference to recommendations 712. According to embodiments, expand icon 914 may be provided such that if a clinician desires to view recommendations for one or more additional or alternative settings adjustments, the expand icon 914 may be activated and the recommendations may be provided on an expanded prompt (not shown).

As may be appreciated, the disclosed data, graphics, and prompt illustrated in graphical user interface 900 may be arranged in any suitable order or configuration such that information and recommendations may be communicated to the clinician in an efficient and orderly manner. The disclosed data, graphics, and prompt are not to be understood as an exclusive array, as any number of similar suitable elements may be displayed for the clinician within the spirit of the present disclosure. Further, the disclosed data, graphics, and prompt are not to be understood as a necessary array, as any number of the disclosed elements may be appropriately replaced by other suitable elements without departing from the spirit of the present disclosure. The illustrated embodiment of the graphical user interface 900 is provided as an example only, including potentially useful information and recommendations that may be provided to the clinician to facilitate communication of a projected impact of a proposed settings adjustment in an orderly and informative way, as described herein.

### Ventilator GUI Display of Expanded Prompt

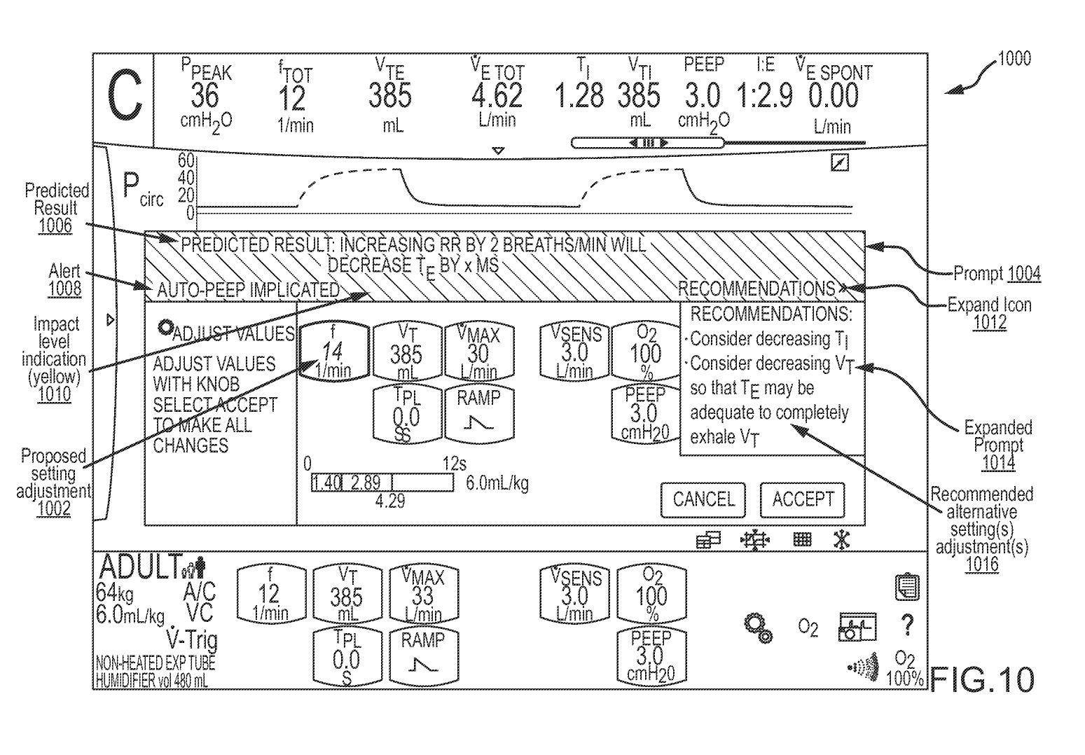

FIG. 10 is an illustration of an embodiment of a graphical user interface displaying a prompt and an expanded prompt identifying a projected impact of a proposed setting adjustment and identifying one or more recommendations for alternative settings adjustments.

Graphical user interface 1000 may display a settings adjustment screen that may provide one or more settings elements, as described above. Upon selection of a settings element, the clinician may input a proposed setting adjustment, e.g., proposed setting adjustment 1002. In the illustrated embodiment, proposed setting adjustment 1002 involves an increase in RR of 2 breaths/min (from 12 breaths/min to 14 breaths/min).

As described above, upon entry or input of proposed setting adjustment 1002, a prompt may be displayed on the GUI, e.g., prompt 1004. Prompt 1004 may further provide a predicted result of the proposed setting adjustment 1002, e.g., predicted result 1006. In this case, the proposed setting adjustment 1002, i.e., increasing RR by 2 breaths/min, may reduce T_{E} by x ms. Specifically, an increase in the number of breaths per minute decreases the amount of time available for T_{I} and T_{E} associated with each breath. As such, according to the illustrated embodiment, predicted result 1006 states “Increasing RR by 2 breaths/min will decrease T_{E} by x ms.”

According to further embodiments, a projected impact may be calculated for proposed setting adjustment 1002. For example, the ventilator may determine that a decrease in T_{E} by x ms may not provide sufficient time to exhale V_{T}, based on collected ventilatory data and/or patient data. As such, the ventilator may determine that Auto-PEEP is implicated by the proposed setting adjustment 1002. As such, the ventilator may issue alert 1008, e.g., “Auto-PEEP Implicated.” Further, the ventilator may provide prompt 1004 in a yellow color to communicate an intermediate impact level of the proposed setting adjustment 1002, i.e., impact level indication 1010.

Prompt 1004 may further provide recommendations for one or more alternative settings adjustments. According to embodiments, expand icon 1012 may be provided such that if a clinician desires to view recommendations for one or more additional or alternative settings adjustments, the expand icon 1012 may be activated and the recommendations may be provided on an expanded prompt, e.g., expanded prompt 1014. For example, based on proposed setting adjustment 1002 (i.e., increasing RR by 2 breaths/min), the ventilator may determine that the clinician wishes to increase ventilation of the patient. However, as indicated above, according to the illustrated embodiment, increasing RR by 2 breaths/min is predicted to decrease T_{E} by x ms. which the ventilator determined implicated Auto-PEEP.

As such, the ventilator may determine alternative settings adjustments that may increase ventilation without implicating Auto-PEEP, e.g., recommended alternative settings adjustments 1016. For example, the ventilator may additionally or alternatively recommend decreasing T_{I}, such that T_{E} may be increased, allowing additional time to exhale V_{T}. Additionally or alternatively, the ventilator may determine that at an increased RR of 2 breaths/min, a decrease in V_{T} may still provide increased ventilation without implicating Auto-PEEP. As such, the ventilator may suggest decreasing V_{T} so that T_{E} may be adequate to completely exhale V_{T} at an increased RR. As should be appreciated, the ventilator may determine any number of additional and or alternative settings adjustments based on a target ventilation change, collected ventilatory data, patient data, etc.

As described above, extended prompt 1014 may be provided adjacent to prompt 1004, along a border of the graphical user interface, near an alarm display or bar, or in any other suitable location. The shape and size of the extended prompt may further be optimized for easy viewing with minimal interference to other ventilatory displays. The prompt 1004 and/or extended prompt 1014 may be provided in a transparent form (not shown), or otherwise, for minimizing distraction, and may be cleared upon clinician viewing. According to the illustrated embodiment, extended prompt 1014 is provided under and adjacent to prompt 1004.

As may be appreciated, the disclosed data, graphics, and prompt illustrated in graphical user interface 1000 may be arranged in any suitable order or configuration such that information and recommendations may be communicated to the clinician in an efficient and orderly manner. The disclosed data, graphics, and prompt are not to be understood as an exclusive array, as any number of similar suitable elements may be displayed for the clinician within the spirit of the present disclosure. Further, the disclosed data, graphics, and prompt are not to be understood as a necessary array, as any number of the disclosed elements may be appropriately replaced by other suitable elements without departing from the spirit of the present disclosure. The illustrated embodiment of the graphical user interface 1000 is provided as an example only, including potentially useful information and recommendations that may be provided to the clinician to facilitate communication of a projected impact of a proposed settings adjustment in an orderly and informative way, as described herein.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the appended claims.

What is claimed is:

1. A ventilator-implemented method for issuing a prompt in response to a proposed setting adjustment during ventilation of a patient, the method comprising:
receiving the proposed setting adjustment;
retrieving a patient diagnosis, at least some collected ventilatory data, and one or more current ventilatory settings; and
determining and displaying a projected impact of the proposed setting adjustment based on the one or more current ventilatory settings, the patient diagnosis, and the at least some collected ventilatory data.

2. The method of claim 1, further comprising:
determining and displaying an impact level of the projected impact, wherein the impact level comprises one of a positive impact level, a negative impact level, and an intermediate impact level based on the patient diagnosis and the at least some collected ventilatory data.

3. The method of claim 1, further comprising:
identifying a target ventilation change; and
determining and displaying one or more alternative settings adjustments to achieve the target ventilation change based on the patient diagnosis, the at least some collected ventilatory data, and the one or more current ventilatory settings.

4. The method of claim 1, further comprising:
receiving a plurality of proposed settings adjustments; and
determining and displaying a combined projected impact of the plurality of proposed settings adjustments.

5. The method of claim 4, wherein an indication is received upon receipt of the last of the plurality of proposed settings adjustments, and wherein the combined projected impact is determined after receiving the indication.

6. The method of claim 1, further comprising:
identifying a target ventilation change;
determining one or more alternative settings adjustments to achieve the target ventilation change based on the patient diagnosis and the at least some collected ventilatory data; and
displaying an icon for accessing the one or more alternative settings adjustments, wherein upon activating the icon the one or more alternative settings adjustments are displayed.

7. A ventilatory system for issuing a prompt in response to a proposed ventilatory setting adjustment during ventilation of a patient, comprising:
at least one processor; and
at least one memory, communicatively coupled to the at least one processor and containing instructions that, when executed by the at least one processor, perform a method comprising:
receiving a proposed setting adjustment;
retrieving at least some collected ventilatory data and one or more current ventilatory settings;
determining a projected impact of the proposed setting adjustment based on the one or more current ventilatory settings and the at least some collected ventilatory data;
determining one or more alternative settings adjustments, comprising:
identifying a target ventilation change; and
determining one or more alternative settings adjustments for achieving the target ventilation change based on the one or more current ventilatory settings and the at least some collected ventilatory data; and
displaying the prompt identifying the projected impact of the proposed setting adjustment and the one or more alternative settings adjustments.

8. The ventilatory system of claim 7, further comprising:
determining an impact level of the projected impact, wherein the impact level comprises one of a positive impact level, a negative impact level, and an intermediate impact level on the patient; and
displaying an indication of the impact level of the projected impact on the prompt.

9. The ventilatory system of claim 7, wherein the prompt further comprises an alert associated with the projected impact.

10. The ventilatory system of claim 7, further comprising retrieving patient data, wherein the patient data comprises at least one of: a patient diagnosis, a patient disability, and a patient post-operative condition.

11. The ventilatory system of claim 10, wherein determining the projected impact of the proposed adjustment is further based on the patient data.

12. The ventilatory system of claim 10, wherein determining the one or more alternative settings adjustments to achieve the target ventilation change is further based on the patient data.

13. The ventilatory system of claim 7, wherein identifying the one or more alternative settings adjustments on the prompt comprises displaying an icon on the prompt for accessing the one or more alternative settings adjustments, and wherein upon activating the icon the one or more alternative settings adjustments are displayed as recommendations on an extended prompt.

14. A graphical user interface for displaying one or more prompts in response to receiving a proposed setting adjustment, the ventilator configured with a computer having a user interface including the graphical user interface for accepting the proposed setting adjustment and for displaying information, the graphical user interface comprising:
at least one window; and
one or more elements within the at least one window comprising at least one prompt element for communicating information regarding the proposed setting adjustment, wherein the at least one prompt element identifies a projected impact of the proposed setting adjustment.

15. The graphical user interface of claim 14, wherein the at least one prompt element identifies one or more recommendations, and wherein the one or more recommendations include at least one alternative settings adjustment.

16. The graphical user interface of claim 14, wherein the at least one prompt element further identifies an impact level of the projected impact of the proposed setting adjustment, and wherein the impact level is one of a positive impact level, a negative impact level, and an intermediate impact level on the patient.

17. The graphical user interface of claim 16, wherein the positive impact level is identified by a green color, the negative impact level is identified by a red color, and the intermediate impact level is identified by a yellow color.

18. The graphical user interface of claim 15, wherein the one or more recommendations are identified on the at least one prompt element by an icon, and wherein upon activating the icon the at least one alternative setting adjustment is displayed on an extended prompt element.

19. A ventilator processing interface for displaying one or more prompts in response to receiving a proposed setting adjustment, comprising:
means for accepting the proposed setting adjustment;
means for retrieving one or more current ventilatory settings and at least some collected ventilatory data;
means for determining a projected impact of the proposed setting adjustment; and
means for displaying a prompt comprising the projected impact.

20. The ventilator processing interface of claim 19, wherein the prompt further comprises one or more recommendations comprising one or more alternative setting adjustments.
